(19) United States
(12) Patent Application Publication
(10) Pub. No.: US 2021/0050086 A1
(43) Pub. Date: Feb. 18, 2021

(54) GENERATING OPTIMISED WORKOUT PLANS USING GENETIC AND PHYSIOLOGICAL DATA

(71) Applicant: FITNESSGENES LTD, Bicester, Oxfordshire (GB)

(72) Inventors: Paul ROSE, Bicester, Oxfordshire (GB); DANIEL REARDON, Bicester, Oxfordshire (GB); ABDULLAH KHAN, Bicester, Oxfordshire (GB); JOHANNES DOEVELAAR, Bicester, Oxfordshire (GB); PLEUNI HOOlJMAN, Bicester, Oxfordshire (GB); STUART GRICE, Bicester, Oxfordshire (GB); SAMANTHA DECOMBEL, Bicester, Oxfordshire (GB)

(21) Appl. No.: 16/964,935

(22) PCT Filed: Jan. 24, 2019
(86) PCT No.: PCT/GB2019/050204

§ 371 Date: Jul. 24, 2020

(30) Foreign Application Priority Data
Jan. 24, 2018 (GB) 1801137.9

(51) Int. Cl. G16H 20/30; G16H 10/60

(52) U.S. Cl. CPC G16H 20/30; G16H 10/60

(57) ABSTRACT

The present invention includes methods and systems for generating an optimised workout plan for an individual based on the individual's genetic, physiological, behavioural and lifestyle data. By taking an individual's genetic and environmental data into account, a workout plan can built from individual exercises and exercise parameters in a way that is optimised for the individual's physiological traits.

### FIELD

The invention relates to methods and systems for generating an optimised workout plan for an individual based on the individual's genetic, physiological, behavioural and lifestyle data.

### BACKGROUND

A person's overall health and fitness are determined by a number of different genetic and environmental factors. Medical history, fitness activity, nutritional choices, air quality, geographic location and mental health, as well as other non-genetic factors including lifestyle choices, access to fitness equipment, monetary considerations, etc. can all have an effect on the individual's overall health and fitness.

However, even with the increasing availability of genetic sequencing and analysis, there still exists no mechanism for an individual to understand how their genetics relate specifically to them, how they interact with environmental factors and each other, and how they may be utilised to best improve their health and fitness.

Existing systems, such as the one described in US 2003/0204412 A1, use feedback data to adjust a wellness plan, interpreted in the context of user input data, but do not use genetic information either to generate the plans in the first place or to inform the modifications to the plans. Genetic descriptions can be an effective way to predict an individual's response to exercise.

Even the systems that do take genetic data into account, such as the one described in US 2016/0217266 A1, assign users with one of a number of pre-defined plans that are designated as being suitable for that user's genetic data. There is no element of personalisation of the plan to a user's individual genetic profile or traits.

Thus, a system is required in which an individual's environmental, genetic and physiological factors are used to generate and tailor a workout plan to the individual to provide guidance relating exercises that will produce the most efficient and effective physiological response in the individual based on the goal they wish to achieve.

### SUMMARY

According to a first aspect of the invention, a computer-implemented method of generating an optimised workout plan for an individual is provided. The method comprises:
receiving genetic data, the genetic data describing a plurality of genetic factors of the individual;
receiving environmental data, the environmental data describing a plurality of environmental factors of the individual;
calculating a trait score for each of a plurality of traits based on the genetic data and environmental data;
defining a trait profile for the individual from the trait scores, said trait profile characterizing the user's physiological response to exercise;
generating an optimised workout plan for the individual by:
categorising days in a given time period into day types according to the training type and trait profile;
for each categorised day, identifying one or more exercises according to the day type and the trait profile;
for each identified exercise, determining one of more exercise parameters according to the individual's trait profile; and
compiling the categorised days, identified exercises and exercise parameters into an optimised workout plan covering the given time period;
transmitting the optimised workout plan to the individual or a healthcare practitioner.

The environmental data may comprise a target number of days per week on which exercise takes place.

Categorising days in a given time period according to a training type may comprise categorising each day according to the type and intensity of exercise to be carried out on the day.

Identifying one or more exercises may comprise querying a database of exercises in which the exercises are categorised according to compatible traits and day types.

After the step of transmitting, the method may further comprise:
receiving feedback data describing a physical condition of the individual;
modifying the optimised workout plan based on the feedback data; and
transmitting the modified optimised workout plan to the individual.

Modifying the optimised workout plan may comprises modifying the exercise parameters.

The feedback data may correspond to a physiological condition of the individual before, during, and/or after performing an exercise.

Modifying the optimised workout plan may further comprise comparing the feedback data to a threshold and modifying the optimised workout plan based on whether the feedback data is above or below the threshold.

Modifying the optimised workout plan may also further comprise modifying the trait profile for the individual based on whether the feedback data is above or below the threshold.

In a specific embodiment, the feedback data comprises heart rate data for the individual and exercise data such that the heart rate data describes the individual's heart rate before, during, and/or after the exercise described by the exercise data. The heart rate data may indicate that the individual's heart rate decreases at a rate below a threshold after a set exercise, and modifying the optimised workout plan may comprise modifying the exercise parameters by increasing the rest time between exercises. Alternatively, the heart rate data may indicate that the individual's heart rate decreases at a rate above a threshold after a set exercise. Modifying the optimised workout plan may then comprise modifying the exercise parameters by decreasing the rest time between exercises.

The threshold for modifying the workout plan may be set according to the trait profile. In the specific heart rate embodiment, the threshold may be set according to recovery and/or lactate clearance traits.

As an alternative, or in addition to modifying the workout plan directly, the method may further comprise updating the trait profile based on the received feedback data. The feedback data may be received from a wearable electronic device.

The optimised workout plan may be transmitted to one or more of: a mobile phone app, desktop app, tablet app, email address, web browser, wearable electronic device, and an exercise machine.

The traits may include one or more of: insulin sensitivity; obesity risk; gut microbiome profile; blood testosterone levels; dyslipidaemia, lactose intolerance, blood triglycerides level, blood glucose levels, oxidative muscle dominance, saturated fat level, satiety, folate metabolism, homocysteine levels, methionine levels, caffeine metabolism, hypertension levels, omega 6 intake or omega 3 to 6 ratio, circadian rhythm, sleep disturbance, trainable VO2 max, salt sensitivity, workout recovery between workout sessions, workout recovery during a workout session, lactate clearance levels, basal metabolism, lean body mass, endurance capability, power capability, conscious restraint, binge eating propensity, emotional eating propensity, eating behaviour and body fat.

The genetic factors may be genetic variants, and the genetic variants may include one or more: polymorphisms; and/or insertions; and/or deletions; and/or gene copy number variants.

The traits characterise biological systems of the user, said biological systems providing a representation of the user's physiological, behavioural and biological propensities and/or health status.

Calculating a trait score for each of the plurality of traits may comprise:
identifying one or more of the genetic factors that are relevant to the trait;
identifying one or more of the environmental factors that are relevant to the trait;
assigning a weighting to each genetic and/or environmental factor, the weighting defining the effect that the genetic and environmental factors have on the trait; and
calculating a trait score for each trait based on the weightings and genetic and/or environmental factors.

The method may further comprise:
receiving feedback data describing a physical condition of the individual;
comparing the feedback data of the individual with feedback data describing physical conditions of a group of individuals, wherein the group of individuals and the individual share a genetic factor;
updating the weighting assigned to the genetic factor based comparison; and
generating a new optimised workout plan for a new individual using the updated weighting.

The exercise parameters may comprise one of more of: number of reps, number of sets, time between sets, rest time, weight to be lifted, distance to be run, distance to be cycled, speed to be run, speed to be cycled, time to run, time to cycle, interval length.

In a second aspect of the invention, a data-processing system comprising means for carrying out the method described above is provided.

In a third aspect of the invention, a computer program product adapted to perform the method described above is provided.

Also described is a method comprising the steps of: identifying one or more traits, said traits being one or more physiological, behavioural and/or biological states of the user; identifying one or more genetic factors of the user that are relevant to one or more of the traits; identifying one or more environmental factors of the user that are relevant to said one or more of the traits; assigning a weighting to each genetic and/or environmental factor for the traits, said weighting defining the likely effect that the genetic and environmental factor has on the traits; calculating a trait score for each trait based on the weightings assigned to each trait, each trait score indicative of the likely biological, behavioural or physiological response of the user to the trait; defining a trait profile for the user from the trait scores, said trait profile characterizing the users likely biological, behavioural or physiological response to a workout plan/fitness programme.

The method provides a mechanism for correlating genetic and environmental factors to provide a workout plan that is optimised for the user. The method determines or identifies a number of traits that are potentially relevant to the goal of the user, such as testosterone level, VO2 max and power ability for a user intending to increase their muscle mass. Genetic and environmental factors relevant to the identified traits are then identified. These factors may be identified from a predetermined listing, or the factors may be determined based on feedback from the user, or based on collated information of an ensemble of users. For example, by analysing users having similar environmental factors, genetic factors influencing traits may be determined. As a specific example, for testosterone genes, genetic variations associated with the ESR1, ACTN3, SHBG1 and SHBG2 are considered relevant genetic factors, whilst body type, body fat %, age, activity levels, weekly active hours and sex may be relevant environmental factors. From these factors, a trait score may be determined. Weightings can be used to provide a statistical weighting to the factors—it can be appreciated that age, sex and activity levels may provide a greater weighting to overall testosterone levels than one or more of the genes, and vice versa.

The trait score is intended to be indicative or define the likely biological, behavioural or physiological response of the user to the trait. Additionally, or alternatively, an external test or result may be used. For the earlier testosterone example a blood testosterone reading may be used to determine a precise trait score.

From the trait scores, a trait profile may be defined for the user. Such a profile acts as an individual indicator of a user's likely biological, behavioural or physiological response to variations in genetic, environmental or behavioural factors and accordingly can be used to characterize a user's response to a workout plan. From this information, a workout plan that is optimised to the goals of the user can be determined.

In an embodiment, one or more of the traits may comprise a plurality of trait bands, each trait band being indicative of particular trait outcome. Furthermore, the step of calculating a trait profile further comprises the step of: assigning a trait score of one or more of the traits to one of the trait bands, depending on the relative strength and directionality of the trait score. By directionality, it is intended to describe whether the trait score is positive or negative, which may correlate to a very high or very low banding suggesting a very high or very low effect of the selected trait within the user's trait profile. Traits can also be unidirectional, or may be defined by any other distribution type.

It can be appreciated that the trait bands may be a grouping of outcomes that describe the user's expected or natural response to the trait being considered. For example, for the trait testosterone 5 bands may be provided, ranging from very low to very high, where each band is indicative of predicted current and/or future testosterone levels at current activity levels and/or at expected activity levels if the user implements the workout plan optimised to their goals suggested by the method.

The method may also include determining one or more recommendations, said recommendations providing an actionable outcome for the user. The recommendations may be determined based on one or more of: trait scores; and/or trait profiles; and/or data elements, said data elements comprising one or more genetic factors, and/or one or more environmental factors and/or one or more goals.

Data elements are intended to provide extra resolution to the recommendations, but are not classed as traits in themselves e.g. one example is a that a recommendation of the Polyunsaturated fat ratio in a diet is determined by the trait defining omega 6 intake (or omega 3 to omega 6 fat ratio) plus a data element describing diet, which means if someone is recommended to increase their omega 3 intake, then a sensible recommendation on how to do this is also based on their diet i.e. if they are vegetarian, a recommendation isn't made to eat more fish.

As an additional example, a recommendation may be provided to the user regarding the total amount of protein that should make up the users diet. This may be provided as a % of food intake, kcal intake, or grams. The recommendation may be given to the user in bands, ranging from very low to very high. The recommendation level or band selected in this instance depends on the trait scores for the traits describing satiety, insulin sensitivity, obesity risk and lean body mass. Non-trait data elements that are relevant to determining the banding or level of recommendation for this example include age of the user and the intermediate/active/ultimate goals of the user.

Like with the trait scores and banding, the recommendations may be weighted depending upon the trait scores or trait bands or data elements which influence the recommendation. For example, a recommendation on whether to supplement the user's diet with vitamin B12 that is dependent on the trait describing folate metabolism and the environmental factor diet is likely to be heavily weighted to diet. It can be appreciated that other weightings may be appropriate for other recommendation.

Weightings and/or recommendations may also be generated based on user data, either from feedback from the present user or from a database having storage of all users response either from surveys or further physical data, that can then be fed back to improve the accuracy of the method.

The traits may include, but are not limited to, one or more of insulin sensitivity; obesity risk; gut microbiome profile; blood testosterone levels; dyslipidaemia, lactose intolerance, resting blood triglycerides level, resting blood glucose levels, oxidative muscle dominance, saturated fat level, satiety, folate metabolism and conversion, homocysteine levels, methionine requirement, caffeine metabolism, drug metabolism, susceptibility to injury, hypertension levels, circadian rhythm, sleep disturbance, sleep cycles, emotional stress levels, physical stress levels, susceptibility to stress, trainable VO2 max, salt sensitivity, workout recovery between workout sessions, workout recovery during a workout session, lactate clearance levels, basal metabolism, lean body mass, endurance capability, power capability, conscious restraint, binge eating propensity, emotional eating propensity, eating behaviour, resting LDL and HDL levels, resting metabolite levels, macronutrient requirements, micronutrient requirements, lean muscle mass %, body fat %, fat usage and metabolism, carbohydrate usage and metabolism, response to omega 6, response to diets types, response to omega 6, biological age, and susceptibility to aging. It can be envisaged that other traits may be identified, particularly traits that are relevant to a workout plan and dependent on a user's genetic and/or environmental condition.

The likely physiological, behavioural and/or biological response of the user to a workout plan indicated by the trait scores, in particular the likely response that the genetic factor has on the traits and/or the likely response that the environmental factor has on the traits, may be an expected physiological/behavioural/biological/genetic or environmental response of a typical user based on comparisons between the user and an existing known user having identical or similar genetic factors, or identical or similar environmental factors or an identical or similar workout plan. Additionally, or alternatively, the likely responses may be predicted responses based on empirical data and/or statistical analysis of prior users. It can also be appreciated that the weightings applied to the genetic factors and/or the environmental factors may be determined in a similar manner using expected or predicted data or statistical analysis.

In examples, the weighting of a trait can be zero, positive or negative. It can be appreciated that negative values may be used that indicate a contra-indication.

The genetic factors may be genetic variants. The genetic variants may include polymorphisms and gene copy number variants.

In examples, the trait profile may characterize the user's biological system. The trait profile may be considered to be the relative levels or propensity to predefined levels or factors.

At least one genetic factor, and/or at least one environmental factor, may be relevant to multiple traits.

The weighting assigned to the genetic factors, and/or the weighting assigned to the environmental factors, may be different for different traits.

A trait score may be calculated from the interaction between one or more genetic factors, and/or between one or more environmental factors, and/or between a combination of genetic and environmental factors, for each or a combination of traits. For example, trait scores for the traits of low satiety and lactose intolerance may be determined or calculated based on a single genetic factor, although it can be appreciated that additional relevant genetic and/or environmental factors relevant to the traits may be added as applicable. The interaction may be one or more of hierarchical, synergistic, additive, subtractive or antagonistic.

A trait score may also be modified by behavioural measures, personality characteristics and/or psychometric analysis, such as the Three-Factor Eating Questionnaire.

A trait score may be modified by direct biophysical data. The direct biophysical data may be data defining the actual biological or physiological response of the user to one or more of said parameters. The direct biophysical data may be provided by a data interface, by a third party biophysical data system or by the user.

The trait profile may be calculated from the hierarchical, synergistic, additive, subtractive or antagonistic interaction of the trait scores.

A separate trait score may be calculated for each expected physiological or biological response of the user to each separate trait.

The trait profile may characterise the users biological system. For example, a trait may be considered the user's propensities to a particular physiological or biological read-out or variable. Typical physiological or biological read-outs can include one or more of: insulin sensitivity; blood testosterone levels; dyslipidaemia and lactose intolerance, although many others can be envisaged.

The step of determining/calculating a trait score may comprise the step of: cross-referencing the one or more genetic factors and/or the one or more environmental factors relevant to said at least one trait and calculating a trait score based on the cross-referencing. This trait score calculation can consider the relative genetic-genetic, genetic-environmental and environmental-environmental interactions. These cross-referencing interactions can take many forms, and may be hierarchical, synergistic, additive, subtractive or antagonistic, for example. Each trait score calculation is typically particular to that said trait and considers known physiological, behavioural or biological variables that impact on that trait.

Traits scores may then be used to calculate a biological system level profile for a user (the biological system models). The step of calculating a biological system model may comprise the step of weighting the one or more traits according to the impact those physiological or biological variables have on each specified biological system (the trait profile for that particular biological system). This results in a specific read-out for each biological system modelled, which, either in combination or individually, can be used to predict an individual's propensity to respond to a particular regime or intervention. Each biological system model read-out may be generated from a single or a number of trait scores. When the biological system model is calculated using the trait profile, the analytical methods for cross-referencing between the trait scores and profiles can take many forms, and may be hierarchical, synergistic, additive, subtractive or antagonistic, for example. It can also be appreciated that the user's biological system model may be calculated from or comprise a plurality of trait profiles.

Trait scores may be used to provide a relative indication of a trait's propensity to affect a particular physiological or biological read-out, or variable. Accordingly, the trait profile at the biological system level can also be considered to be a relative indication of a user's overall physiology or biology, and ultimately their propensity to respond to a particular regime.

A trait profile may be calculated by weighting the trait scores according to the goals of the user.

In examples, the weighting for trait score of a trait in a trait profile for a particular biological system can be zero. This means that a selected trait may provide no indication of a user's propensity to a particular variable or regime, or that any effect of the trait for that biological system does not differ from the default or reference position. It can be appreciated that negative values may be used that indicate a contra-indication.

In examples, traits may initially have a default trait score indicating the average predicted response of a defined average user to the trait. The default trait score can then be updated with the trait score for a specific user. It can be appreciated that the average user may be selected or determined to match the gender, activity level, age, race etc. of the user. An average user may be a male, aged 25 to 40, having moderate exercise and activity levels and a moderate diet. It can be appreciated that the average user may be a construct used for the statistical model.

By utilising such default scores, in the case of missing or incomplete genetic and/or environmental factors, an estimated trait score can be used to ensure that a regime that requires a value for a trait score can be included in the determination step or process.

The biological system models may be used to characterise the users overall physiology or biology. In examples, typical biological systems can include one or more of: metabolism, skeletal muscle system, body fat, liver function, oxygen transport, circulatory system, central nervous system, metabolome, epigenome and gut environment, although many others can be envisaged.

The biological system models may be considered to be a representation of an individual's physiological and biological propensities and health status. Such models may then be used to help predict/identify how an individual will respond to different health, nutritional and/or fitness programs, and, importantly, to match them to the optimal system for their goals and/or needs.

In examples, different health, nutritional and/or fitness programs may be determined for different biological system models. Typical queries relating to health, nutritional and/or fitness programs may include: ‘How many calories should I consume?’, ‘What type of exercise should I do?’, ‘How frequently should I train?’, ‘What proportion of protein should I eat?’, although many others can be envisaged.

In examples, different workout plans can be matched to users goals or needs. Typical goals or needs may include fat loss, muscle building, endurance training, cardiovascular improvement, rehabilitation from illness/disease, disease prevention, although many others can be envisaged.

In examples, the method may comprise the steps of matching said trait scores to one or more default, or reference traits. The reference traits may be associated with reference trait profiles, said reference traits having reference trait scores defining the response of a reference group of users to a physiological or biological variable. Accordingly, the reference trait profiles may provide an overall reference response of a particular group of users. By utilising reference traits and reference trait profiles for large groups of users, statistical analysis and learning (machine or otherwise) can be used to match or predict the likely response of the user based on the difference between their traits and trait profile and the reference traits and trait profile. Artificial intelligence, such as IBM Watson® may be employed to predict the likely response of the user and/or to predict an optimal workout plan. As the reference trait database grows, the accuracy of the matching between the predicted response and the actual user response should improve.

In examples, the genetic and environmental factors may be scored according to ensemble research to determine a genetic or environmental factor score assigned weighting/value indicative of the user's genetic or environmental factor compared to the ensemble research. The trait scores may also be determined or modified using the factor score of each one or more genetic and environmental factor.

In another example, the method may include the steps of: identifying a fitness goal of the user; querying a database of traits relevant to the said goal; and prioritising the traits identified by the database for the said goal. The prioritising can comprise the steps of: adjusting the trait scores of the user corresponding to the traits identified by the database according to weighting factors stored within the database; and updating the trait profile based on the adjusted trait scores.

Utilising goals allows the method or system to tailor a health, nutritional and/or fitness regime to match the goals and/or needs of the user. It can be appreciated that many users have a goal or ambition that they wish to achieve from a health, fitness or nutritional regime. It may be appreciated that the optimum regime for the user's goal may not be the same at any point in time, if the user's goal is incompatible or unsuited (perhaps temporarily) with the user's trait profile. For example, a pre-existing health condition or situation may need to be addressed first to enable the ultimate goal to be achieved more effectively and/or safely. In such a scenario, the method or system can select a regime that it considers to be in the best interests of the user. This may be considered to be an active goal for the user, which may differ from the user's ultimate goal. For example, the ultimate goal of the user may require a base level of strength or physical fitness to achieve, therefore the active goal determined by the method or system instead provides a plan for the user to first reach the prerequisite base levels for the ultimate goal to be achieved.

It can be appreciated that having the user provide a feedback response regarding the health, nutritional and/or fitness regime allows the databank of trait profiles matched to health, nutritional and/or fitness regimes to be improved for future users. Additionally, based on the feedback response, the trait scores, trait profiles or, where applicable, the factor weightings can be altered and the system or method reanalysed to update the health, nutritional and/or fitness regime suggestion. The feedback response may be collated within the system or a databank, which may be a sandbox environment. The feedback response may result in one or more updated environmental or genetic factors. The trait score of the one or more traits may be altered based on the feedback.

In examples, feedback response can be measured using questionnaire, app technology, wearables, lab based studies and clinical trial, although many others can be envisaged.

In response to feedback the trait score may be altered within a sandbox database until the overall predicted response of the user matches the feedback response, to test the overall higher impact of the change. This allows the system or method to adapt and analyse and update the associated scores and rules without potentially negatively affecting the user. Once the sandbox analysis is complete and verified, the updated trait values may be applied to the traits of the user within a live database.

The genetic factors, environmental factors and traits may define a profile for the user. The genetic factors may be identified from one or more of: a laboratory DNA test; a user DNA test; or medical laboratory data. The environmental factors may be identified, for example, from one or more of: health and fitness applications; questionnaires; mobile applications; laboratory data; health and fitness monitors or a computing device.

In examples, the user may provide a feedback response regarding the workout plan. The user feedback may be provided by an interfaced system, optionally a third party system, although it can be appreciated that a complementary system provided by the provider of the method may be used. Additionally or alternatively, the feedback response may be used to update the weightings of one or more of the environmental factors (or conceivably the genetic factor weighting). The trait score of the one or more traits may also be altered based on the feedback.

Additional genetic and/or environmental factors may also be later determined for certain traits. As necessary, such additional factors may be used to update the weighting of the factors (and also therefore the trait scores and trait profile).

The trait score may be altered within a sandbox database until the overall predicted response of the user matches the feedback response. The trait values may then be supplied to the trait profile within a live database.

The genetic factors, environmental factors and traits may define a profile for the user.

The genetic factors may be identified from one or more of: a laboratory DNA test; a user DNA test; or medical laboratory data. Such data may be provided from third party companies via data downloads, APIs or other known data transfer mechanisms.

The environmental factors may be identified from one or more of: health and fitness applications; questionnaires; mobile applications; laboratory data; health and fitness monitors, user location, user psychological history, location conditions, or a computing device. A user's medical history may also be considered.

The method may comprise the step of exporting information representative to said trait profile to a third party server or external server. Such information may be considered to be a digital fingerprint representation of the trait profile, or the trait profile itself. The digital fingerprint may be used to ensure an additional level of security for the trait profile. The digital fingerprint may restrict access to the trait scores and the other raw data such as the weightings, for example, restricting 3rd party access to the raw data, whilst allowing interfacing with 3rd parties via an API (application programming interface). The method may further comprise the step of providing advertising to the user tailored to the trait profile.

The method may further comprise the step of interfacing with external data sources to supplement and/or incorporate user data within the trait profile. The interface may be via a mobile application.

The external data sources may interface with a server implementing the method such that the server provides a guided manual for analysis of the external data. Accordingly, such an example may provide a black-box arrangement where data is provided to the server from external data sources, with workout plans and/or other requested data provided by the server. In this manner, the server may be used to provide restricted access to one or more trait or environmental or other user specific data to allow additional applications to be run based on the data. It can be envisaged that such other apps may utilise for example the caffeine metabolism trait score to determine which coffee bean to advertise/recommend to an individual, for example.

It can be envisaged that such other apps may utilise for example the insulin sensitivity trait score, in addition to other factors, to determine a patient's risk of developing diabetes, for example.

According to a second aspect of the invention there is provided a system for carrying out the method described in the first aspect.

As broadly defined herein, aspects of the invention may define a method having at least some of the following steps: identifying one or more genetic variants (including, but not limited to, polymorphisms and/or insertions and/or deletions and/or gene copy number variants) of a user, with each of these genetic factors being assigned a weighting/value that is used to calculate the likely, typically the expected or a predicted, response on a physiological, behavioural or biological parameter, called a ‘trait’. Each genetic factor can impact one or multiple traits. The weightings assigned to the genetic factors can also be different for different traits. This may allow the weighting assigned to each genetic factor to be different when applied to different traits.

Identifying one or more environmental factors of a user, with each environmental factor being assigned a weighting/value that is used to calculate the likely, typically the expected or a predicted, response on a trait can then be performed. Each environmental factor can impact one or multiple traits. The weightings assigned to the environmental factors can also be different for different traits. This may also allow the weighting assigned to each environmental factor to be different when applied to different traits.

Calculating a score for a ‘trait’, which is a defined physiological, behavioural or biological read-out, using the combined genetic and environmental weightings/values that have been scored for that trait, to generate a trait score can then be performed. The trait score calculation may consider the relative genetic-genetic, genetic-environmental and environmental-environmental interactions. These interactions may be hierarchical, synergistic, additive, subtractive or antagonistic, for example. Additionally, a predicted trait score may be modified by direct biophysical data, where known.

Trait profiles may then be used to calculate models of an individual's biology systems and ultimately an individual's health and fitness status (the ‘biological system’ level). Each biological system model may be generated from a number of trait scores (the trait profile for that biological system). When a biological system model is calculated using the trait profile, the interaction between different trait scores may be hierarchical, synergistic, additive, subtractive or antagonistic. The biological system models can be used, either in combination or individually, to help, or to solely, predict how an individual will respond to different health, nutritional and/or fitness programs, and to match them to the optimal system for their goals and/or needs.

This approach considers the collective effects of both genetic and environmental factors in determining health, nutritional and/or fitness regimes for a user. By utilising both factors in combination, the ensuing biological system models, which are predicted by the trait profile, may be used to calculate a more personalised and accurate biological and health profile of the user. An additional key part of this method is that trait scores can also be used in combination to infer a higher level of understanding of an individual, and ultimately allow for more targeted and accurate recommendations.

The present disclosure describes providing a system and method which uses a combination of user-specific relevant genetic, environmental and lifestyle data to define a number of “traits”, which is a defined physiological, behavioural or biological read-out, and then using these traits to model a smaller number of higher-level biological systems. When the overall system or method is queried as to what health regime, fitness regime or diet would meet a particular desired goal, an algorithm or artificial intelligence system may be used to match fitness/training/diet advice with the user's specifically defined biological system and trait profile, including the compounding effects of trait-trait interactions at the biological system level.

The examples described herein relate to collecting and analysing user-specific genetic and environmental data to develop comprehensive, personalised diet and fitness programs for helping a user achieve their desired goal or a better health status. The user-specific data may be collected from a variety of sources, including, but not limited to, genetic testing, lab testing, nutrition information, physiological tests, health tests, metabolic testing, psychological testing, mobile health devices worn by the user and applications through which the user manually inputs information.

The user-specific data is collected and analysed together, based on knowledge of the interrelationships between general genetic and environmental data, to develop a user profile with personalised diet and fitness programs that are targeted to improving specific areas of the user's health by implementing changes in exercise, nutrition, environment etc. These plans are highly personalised as they are based upon a person's own genetic code and their environmental condition/lifestyle choices.

A user interested in finding a fitness plan that is optimal for their own personal genetic make-up and their health and lifestyle history, and one that may be targeted at achieving a specific goal, may submit a genetic sample, for example saliva, for genetic information extraction. The received sample containing genetic material may be processed to produce a genetic readout suitable for analysis at a databank. Stored algorithms having parameters which depend upon genetic features may be applied to the data to produce an output to characterise a user in terms of their genetic factor score.

Further, the system uses the genetic and environmental data to define a number of “traits”, or propensities towards particular phenotypic expressions, and then uses these traits to model a smaller number of higher-level biological systems. A trait, as defined here, may be a physiological, behavioural or biological metric based on genetic and/or environmental factors; for example, insulin sensitivity; obesity risk; gut microbiome profile; blood testosterone levels; dyslipidaemia, lactose intolerance, resting blood triglycerides level, resting blood glucose levels, oxidative muscle dominance, saturated fat level, satiety, folate metabolism and conversion, homocysteine levels, methionine requirement, caffeine metabolism, drug metabolism, susceptibility to injury, hypertension levels, circadian rhythm, sleep disturbance, sleep cycles, emotional stress levels, physical stress levels, susceptibility to stress, trainable VO2 max, salt sensitivity, workout recovery between workout sessions, workout recovery during a workout session, lactate clearance levels, basal metabolism, lean body mass, endurance capability, power capability, conscious restraint, binge eating propensity, emotional eating propensity, eating behaviour, resting LDL and HDL levels, resting metabolite levels, macronutrient requirements, micronutrient requirements, lean muscle mass %, body fat %, fat usage and metabolism, carbohydrate usage and metabolism, response to omega 6, response to diets types, response to omega 6, biological age, and susceptibility to aging.

This approach considers the effect of both genetic and environmental (which includes lifestyle) factors in determining a health regime for a user. By utilising both factors, the trait profile may provide a customised profile of the user and their likely response to variables such as diet, exercise, medication or the like. The trait profile can consider the relative interactions between genetic-genetic factors, genetic-environmental factors and environmental-environmental factors. This ensures that a broader perspective and consideration is given to selecting a health regime that takes account of both genetic and environmental relevant factors, rather than purely focussing on, typically, genetic issues as has been generally the case. The workout plan may include one or more of fat loss plans, muscle building plans, strength training plans, endurance training plans, sporting performance plans.

Additionally, aspects of the present invention may include a compliance factor, wherein the method further comprises one or monitoring sensors or devices that are configured to analyse a user's compliance with the determined workout plan. Based on the information collated by the sensors or devices, the workout plan may be updated or adjusted in real time to either ensure that the original goals of the user are met or alternatively to provide an alternative intermediate or active goal for the user to aim towards. This may be useful for the user to allow them to understand or appreciate how deviating from the suggested recommended workout plan influences their chance of achieving their goals.

There may be provided a computer program, which when run on a computer, causes the computer to configure any apparatus, including a circuit, controller, sensor, filter, or device disclosed herein or perform any method disclosed herein. The computer program may be a software implementation, and the computer may be considered as any appropriate hardware, including an analyser, a microprocessor, and an implementation in read only memory (ROM), erasable programmable read only memory (EPROM) or electronically erasable programmable read only memory (EEPROM), as non-limiting examples. The software implementation may be an assembly program. A user interface may be provided to allow for the inputting of information by the user—such interface may be a webpage, application or hardware based interface.

The computer program may be provided on a computer readable medium, which may be a physical computer readable medium, such as a disc or a memory device, or may be embodied as a transient signal. Such a transient signal may be a network download, including an internet download.

It can be appreciated that, although certain examples and embodiments described above have been primarily described with respect to a single aspect, the features described are also applicable to the other aspects defined herein.

These and other aspects of the invention will be apparent from, and elucidated with reference to, the embodiments described hereinafter. The above discussion is not intended to represent every example embodiment or every implementation within the scope of the current or future Claim sets. The Figures and Detailed Description that follow also exemplify various example embodiments. Various example embodiments may be more completely understood in consideration of the following Detailed Description in connection with the accompanying Drawings.

### BRIEF DESCRIPTION OF THE DRAWINGS

Embodiments will be described, by way of example only, with reference to the drawings, in which:

FIG. 1 shows a high-level system diagram of the disclosed invention, identifying the primary stages and directional throughput.

FIG. 2 shows the Genetic and Environmental Data Collection environment and some of the collection mechanisms that may be used within an embodiment of the system shown in FIG. 1.

FIG. 3 shows an embodiment compatible with FIGS. 1 and 2 of a mechanism for calculating the trait scores for a specific user based on the genetic and environmental data collected.

FIGS. 4a-c shows a preferred detailed embodiment for a Trait and Biological System Model described in FIGS. 1 to 3.

FIG. 5 shows an embodiment of the system of FIG. 1 where bandings and recommendations are shown.

FIG. 6 shows an example of a user interface according to an embodiment of the disclosed invention.

FIG. 7 shows a logical overview of the method and system for generating optimised workout plans of the present invention.

It should be noted that the Figures are diagrammatic representations and not drawn to scale. Relative dimensions and proportions of parts of these Figures have been shown exaggerated or reduced in size, for the sake of clarity and convenience in the drawings. The same reference signs are generally used to refer to corresponding or similar features in modified and different embodiments.

### DETAILED DESCRIPTION OF EMBODIMENTS

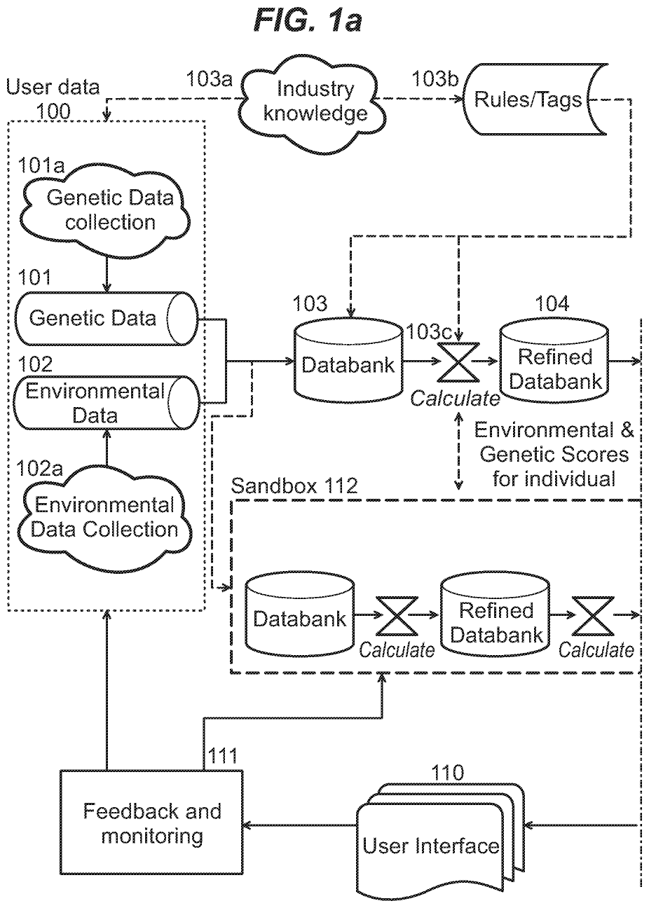

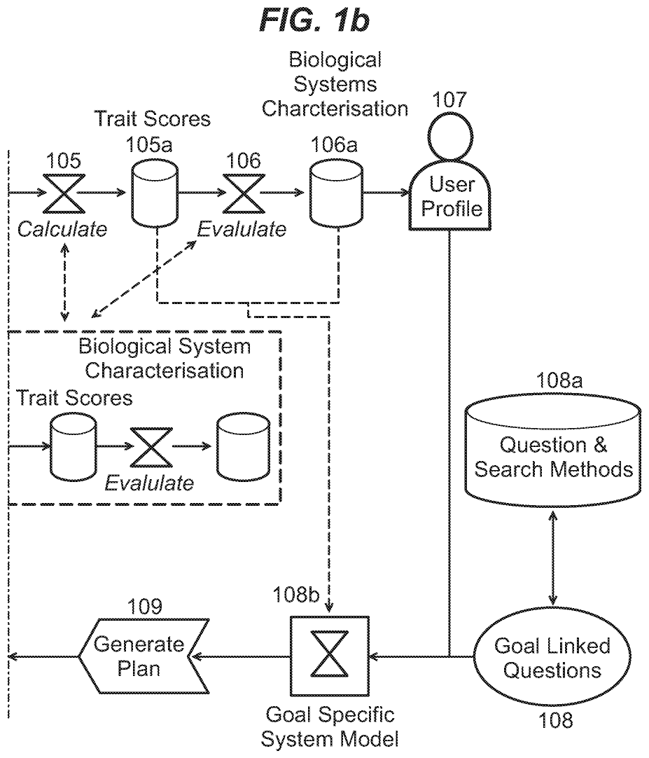

FIG. 1 shows a high-level system diagram of the invention, identifying the primary stages and directional throughput. Dotted lines are used to represent review stages that are updated as the dataset increases.

The following labels within FIG. 1 are defined as:
101 Genetic Data—Digital information regarding the genetic make-up of a user which defines the genetic potential of a user carried in the base sequence of their DNA according to the genetic code.
101a Genetic Data collection—DNA collection which includes, but is not limited to, a saliva sample home collection kit and instructions.
102 Environmental Data—Digital information regarding the health, lifestyle and wellbeing of the individual which may include, but is not limited to, height, weight, age, blood pressure, body fat percentage and fitness activity.
102a Environmental Data collection—Multiple methods of inputting environmental data into the system which may include, but are not limited to, health monitoring applications or devices, questionnaires and professional health data.
103 Databank—a database that contains genetic and environmental data which is linked with the demographic data of an individual prior to analysis and characterisation.
103a Industry Knowledge—Academia and knowledge used to characterise the user based on analysis of their genetic and environmental data.
103b Rules/Tags—Rules and tags extracted from the source/industry knowledge that is used to characterise the databank.
103c Algorithm—An algorithm to tag and categorise the environmental and genetic data, using the defined rules/tags, 103b, to further define and categorise a user.
104 Refined Databank—A database containing further defined and categorised genetic and environmental information about a user based on the data collection, analysis and calculation performed on the initial data within 103. The refined databank contains highly user-specific genetic and environmental scores.
105 Trait Scoring Algorithm—An algorithm used to produce a physiological or biological readout, a “trait score”, for the individual based on combinations of the environmental and genetic scores of the user.
105a Trait Scores—A quantitative physiological or biological readout for one or more traits of a user based on genetic and environmental data, for example, resting blood triglycerides level.
106 Evaluation—Means of characterising biological systems of a user based on the trait scores 105a.
106a Biological System Characterisation—A readout for a physiological understanding of defined biological systems of a user, based on a combination of traits that represent the trait profile for that biological system.
107 User profile—Detailed, annotated understanding and characterisation of the user which is created by utilising customised algorithms 105 106 and the data within the refined databank 104 which leverage understandings of the relationships, the interactions and the compound effects between the genetic, fitness and environmental data.
108 Goal Linked Questions—A health, fitness and/or diet goal for a user such as “increase endurance”, “build muscle” or “lose fat”.
108a Question and Search Methods—A database of fitness goals and diet goals, which are tagged with the necessary search information to find, order and prioritise relevant traits within the biological systems to give the appropriate diet/fitness recommendations.
108b Goal-specific Trait and Biological System Model—An algorithm used to modify scores based on weightings of traits associated with particular goal-linked questions and their effects on the biological systems to produce a readout.
109 Generate Plan—User-specific workout plans are generated based on the outputs of the trait and biological system model.
110 User Interface for delivery of plans to User—user interface to deliver diet, fitness and workout plans as well as translated information on user profile to the user via a graphical user interface.
111 Feedback and monitoring—information on user progress is returned via online, mobile or third party surveys, applications or devices.
112 Feedback Analysis—a sandbox environment uses user-reported data to continually assess and refine the model, test the calculations and evaluate outputs using updated and real-time data.

FIG. 1 illustrates an embodiment of the system for collecting and analysing user-specific data 100 to develop comprehensive personalised diet and fitness programs. In this embodiment, user-specific genetic and environmental data 100 is collected from multiple sources 101a and 102a such as:
Genetic Tests
Mobile health monitoring applications
Questionnaires

User data, also called factors, 100 is collected and stored in the central databank 103, the databank 103 generally contains genetic 101 and environmental 102 factors/information for a specific user. But within the databank at this stage, there may be no weightings or defined characterisation of the user. The received genetic data/factors and environmental data/factors, 101 102 is collected within the databank which stores all relevant information on the user to be defined in the model. The genetic data 101 is assessed to give a very high level overview of the user, for example, “user x has gene y and specific variant z in regards to gene y”. Further, the information within databank 103 is used to identify the presence or absence of certain specific genetic features or markers related to diet, fitness and health.

Further analysis then takes place based on the interrelationships between genetic and environmental data to characterise the user based on the interrelationships and compound effects defined by a combination of the rules/tags 103b, the industry knowledge 103a and the user-specific data 100 within the databank 103.
103a “industry knowledge” can include information from academia, health care professionals, research scientists, health and fitness applications etc. and is used to create rules/tags 103b. These rules/tags are used to categorise and sort the genetic and environmental data in the databank 103 into the refined databank 104.

In one embodiment, after analysis and computing of a user's genetic and environmental data 100 in combination with the industry knowledge 103a and rules/tags 103b, a more defined understanding and characterisation of the user will be present and is stored within the refined databank 104.

In this embodiment, the refined databank 104 typically contains further defined and categorised genetic and environmental information about a user based on the data collection, analysis and calculations performed 103c. The refined databank 104 can contain highly user-specific scores and weightings, based on genetic makeup and environmental data.

Once the user has been characterised within the refined data bank 104, a trait scoring algorithm 105 uses the data in order to analyse, define and generate a more specific user trait profile 107.

In particular, trait scores 105a, which define the biological or physiological response of the user to traits can be calculated. Such traits include but are not limited to one or more of: insulin sensitivity, obesity risk, gut microbiome profile, blood testosterone levels, dyslipidaemia, lactose intolerance, resting blood triglycerides level, resting blood glucose levels, oxidative muscle dominance, saturated fat level, satiety, folate metabolism and conversion, homocysteine levels, methionine requirement, caffeine metabolism, drug metabolism, susceptibility to injury, hypertension levels, circadian rhythm, sleep disturbance, sleep cycles, emotional stress levels, physical stress levels, susceptibility to stress, trainable VO2 max, salt sensitivity, workout recovery between workout sessions, workout recovery during a workout session, lactate clearance levels, basal metabolism, lean body mass, endurance capability, power capability, conscious restraint, binge eating propensity, emotional eating propensity, eating behaviour, resting LDL and HDL levels, resting metabolite levels, macronutrient requirements, micronutrient requirements, lean muscle mass %, body fat %, fat usage and metabolism, carbohydrate usage and metabolism, response to omega 6, response to diets types, response to omega 6, biological age, and susceptibility to aging. It can be envisaged that other traits may be identified, particularly traits that are relevant to a workout plan or fitness programme and dependent on a user's genetic and/or environmental condition.

The user data 100, in particular the trait scores 105a are analysed 106 to generate trait profiles 106a which leverage understandings of the relationships, the interactions and the compound effects between the genetic and environmental data in order to generate a user profile 107. This is further explained within the trait scoring section below.

In some embodiments, the user profile 107 may be queried with goal related questions 108. In one embodiment, a goal of the user could be, but is not limited to, ‘building muscle’, ‘losing fat’, ‘improving endurance’, ‘improving strength’ and ‘improving cardio-vascular capability’ etc. The relevant traits, biological systems and their weightings and hierarchies are specific to the goal, and subsequently the goal related question 108, of the user. For all goal related fitness and diet based questions, the questions can be tagged with the necessary search information to find, order and prioritise relevant traits within the biological systems. This information is then stored within the question and search methods database 108a.

One potential use for the user profile 107 is to allow a user to export the user profile to additional (3rd party) applications or software. This ensures that the genetic and environmental factors that were used to generate the user profile 107 are not transferred to the external software, but the overall profile can still be used to generate other useful data or recommendations.

Certain traits may not be required for computing recommendations, as the traits and systems that are modelled are dependent on the user's goal. The system is given knowledge, either known or learnt, of the relevant traits and biological systems that are relevant to the goal of the user and the system subsequently selects said relevant traits and biological systems to be processed and analysed in the trait and biological system model. The weighting provided to the traits may be altered or reordered based on the knowledge obtained by the system. This, in turn, effects the trait profile generated for the user and the workout plan and/or recommendations provided by the system.

Trait scores 105a and biological system characterization (also referred to as trait profiles) 106a are analysed within the trait and biological system model 108b using customised algorithms or AI systems which leverage understandings of the relationships, interactions and compound effects between the traits and biological systems specific to the goal of the user. These compound relationship weightings based on the specific user profile 107 and goal 108 are then used to develop the optimised workout plans 110 for the user.

The output of the trait and biological system model is used in conjunction with a ‘fetch’ process 109 that matches the relevant trait scores within the relevant biological systems to appropriate schedules, exercises and exercise parameters. These scores are used to query databases comprising exercise and exercise parameters and fit the identified exercises into a customised schedule as described in more detail below.

The user may also be provided with a personal profile and online interactive graphical interface with one or more mobile applications/devices to track and monitor their progress throughout their plans. As the user progresses through their health regime and experiences changes in their overall fitness and health, their diet, fitness and plans may then be updated by re-running the traits and biological system model in a sandbox environment 112 to produce updated scores and subsequently new and updated diet, fitness and plans based on the progress data reported by the user 111. In a similar manner to that described above, the sandbox 112 may have a sandbox databank 103′ and a refined sandbox databank 104′, sandbox calculation 103′, sandbox trait scoring algorithm 105′, sandbox trait scores 105a′, sandbox analysis 106′ and sandbox trait profiles 106a′. These sandbox elements 103′-106a′ operate in an analogous manner to corresponding elements 103-106a, but allow the system to analyse the effect of changes in the progress data reported by the user 111 and to feed these back into the live corresponding elements 103-106a as necessary. The use of feedback to refine the generated workout plans and user trait profiles is described in more detail below.

Mobile health devices, such as wearable fitness trackers, smartwatches or smartphones, and other applications may continue to be utilised to report new user data once the user has begun to implement the workout plan, and this new data can then be used to refine the generated workout plans and user trait profiles, as described in more detail below.

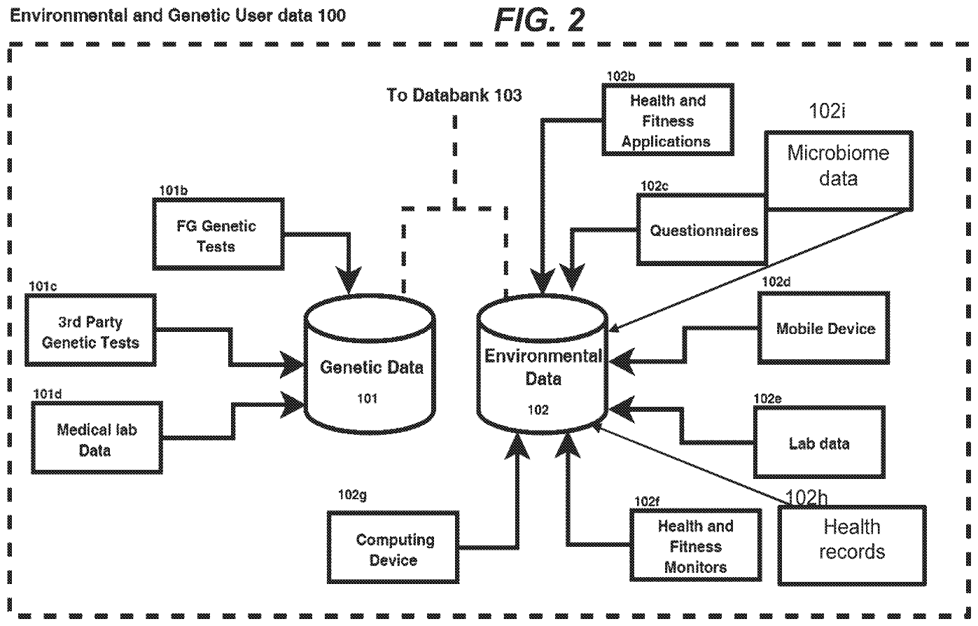

In one embodiment, as seen in FIG. 2, user-specific genetic 101 and environmental data 102 are collected from genetic 101a and environmental 102a collection sources. These sources may include, but are not limited to:
Genetic Data 101:
   DNA test 101b
   DNA collection tests 101c
   Medical genetic lab data 101d
Environmental Data 102:
   Health and fitness applications 102b
   Questionnaires 102c
   Mobile Devices 102d
   Environmental lab and medical data 102e
   Health and fitness monitors 102f
   Computing devices 102g
   Health records 102h
   Microbiome data 102i

In another embodiment, the data sources which collect and transmit data on the user may include, but are not limited to:
BMI monitor
Glucose monitor
Blood pressure monitor
DEXA scans
FitnessGenes user questionnaires
3rd party health and fitness questionnaires
Psychological or behavioural questionnaires or tests
General health check reports from medical professionals
Pedometers
Nutritional tracking applications—e.g. MyFitnessPal™
Apple Health™/Samsung S-Health™
FitBit™

These devices/apps are only exemplary, and multiple other genetic, fitness and environmental monitoring devices may be used to generate user data and transmit data directly to the databank 103. Many wireless health devices have open application programming interfaces (APIs) which allow them to be easily integrated with a system running on the front-end cloud server that will compile, analyse and display the data.

In addition to the devices, additional user data 100 can be collected in the form of genetic data 101 from a genetic report from either first or third party 101b 101c or lab results from lab tests 101d that the user has undergone.

Further, mobile devices and health monitors 102d 102f can be configured to continually collect and report real-time environmental data to the databank so the user profile can be continually updated to provide temporally relevant information about the user's diet and fitness progress.

In one embodiment, a trait is an indication of an individual's physiological or biological properties that have a potential effect on the individual's physiological response to exercise, e.g. resting blood triglycerides level, resting blood glucose, average blood testosterone level, oxidative muscle dominance, body fat, etc.

Even without any genetic or environmental data added to the system, the system can be initialised so that the trait gives a default score. This can be, for example, the average for the general population. Each trait score may be calculated/modified by the genetic and environmental data that has been collected for an individual user.

How the environmental and genetic factors modify a trait can be highly complex and can require meta-directional analysis which allows for compound gene-environment interactions.

Further, each algorithm that calculates each individual trait generally differs with concentration, transformation, and model-based integrations being used to standardise data. Once the environmental and genetic data has been inputted, modelled and characterised within the algorithms, the trait score can then be modified, giving a personalised readout for that specific trait.

As multiple data types may be collected from different sources, there may be redundancy in the data, with some data modifying or overriding other data points. For example, questionnaires that ask individuals to define their body composition, and enter values for their waist circumference, height and weight. This information can be used to predict body fat percentage but may be overwritten/overridden when data defining actual body fat percentages (from more accurate scanning technologies) is present.

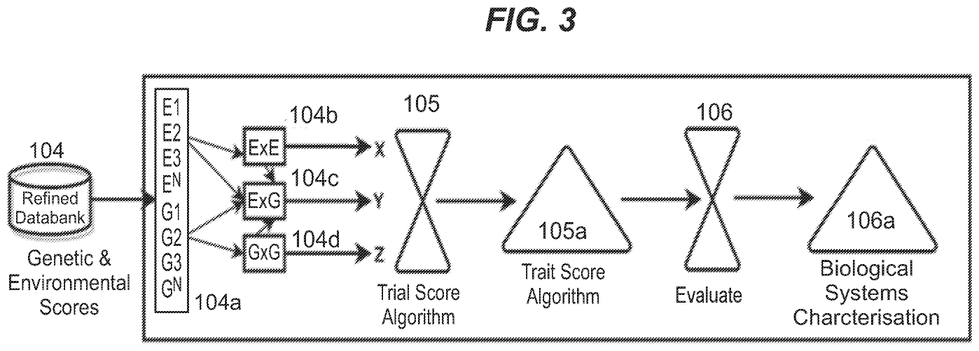

Further, as shown in FIG. 3, selected environmental and genetic data points can be integrated and used to modify the trait score 105a to personalise the readout for an individual. The calculations take into account genetic interactions (G×G) 104d, environmental interactions (E×E) 104b and gene-environment interactions (G33 E) 104c. Algorithms may involve concentration, transformation, and model-based integrations, as they may deal with multiple types of data.

In this embodiment, trait scores give readouts for a physiological understanding of biological systems 106a. One example of the analysis of the biological systems 106a can include, in an embodiment, analysing the trait scores 105a and aligning the trait scores 105a into one or more trait bands. The trait bands provide a series of bandings or categories that the trait scores align against. Each trait band is indicative of a particular trait outcome. For example, trait scores may be deemed or aligned to be very high, high, medium, low or very low for a particular trait. Such bands provide an indication of the relative expression of the trait. Directionality, namely whether a trait contributes or prevents/acts-against an outcome can also be taken into account.

For example, for the trait testosterone five bands may be provided, ranging from very low to very high, where each band is indicative of predicted current and/or future testosterone levels at current activity levels and/or at expected activity levels if the user implements the workout plan suggested by the method.

The use of bandings rather than precise values helps to prevent individual results from skewing recommendations. Continuing with the testosterone example, a very high or very low testosterone trait score may impact upon the method rejecting an otherwise suitable workout plan or may also have the effect of suggesting a routine based on the very high relative trait score of a single trait, rather than the combination of traits, which is the intent of the system.

Table 1 details the logic built for the testosterone trait. Each row details a rule that 1) has an assigned gender, 2) has a series of condition rules that comprise of ‘Data Elements’ and results/values (‘Result High’ and ‘Result Low’), and 3) a rule score of that condition set (‘Rule Value’). Conditions rules are linked together via logic, and any number of condition rules can be applied to a condition set assigned to a rule. The rule value of each rule is taken into the trait calculation if the conditions of the rule are satisfied. The values taken forward are then used in the trait calculation. Rules values can be added together, or can be entered into more complex calculations. In this example, the values are added together. The ‘rule action’ details where the information from a rule is enough to satisfy the trait. If satisfied an ‘Exit’ action is applied.

For example, in the testosterone trait score calculation example, if the system encounters the factor or data element of the user being female then the trait score is null and the trait calculation ends. Similarly, if the (male) user provides an external testosterone blood test level as a data element or factor, then the level of the testosterone blood level result is compared to the conditions according to the banded levels shown. For example, a blood testosterone level of between 0 and 200 results in a trait score of −5.5. Here, because the data input is a known accurate reading, the trait calculation ends.

For rules 7 to 9 of Table 2, a genetic data element is analysed to determine which gene phenotype is expressed. For types TT or TC, the rule value returns a score of 0.0 and the calculation continues. For type CC, a score of 1.0 is (in this example) added to the current or a base trait score.

Similarly, data elements may be combined. This is shown in rules 19, 21, 24, 25 and 27 where the data element relating to BODY Type is linked with the data element BODYFAT. In the example shown, the body type (which is self-reported by the user) is used if the data element BODYFAT is unavailable. It can be appreciated that the bodyfat % data element is generally a more accurate data element for testosterone calculation than body type. Other trait score contributing factors shown in table 2 relate to age, activity and the number of active hours per week (Met_hours). From a combination of the condition rules a trait score can be determined for each trait. It can be appreciated that although the above relates to the testosterone trait, similar conditions and condition sets can be generated and used for other traits.

The trait information may be informative for scientists and healthcare professionals; but in this embodiment they do not by themselves offer recommendations. The trait scores are used in combination to infer higher level understanding of an individual's specific biological systems 106a, and ultimately may allow for actionable recommendations to be made.

It can be envisaged that the trait information could be provided to external parties in a controlled ‘black-box’ arrangement where the system or method compiles information provided to and supplied from the system or method from third parties, whilst shielding such third-parties from the trait-trait interaction and scoring definitions.

Implementation options include:
The genetic testing platform may not limit the number of traits in the system.
Traits may be modified, or even be split into multiple traits.

TABLE 1
Logic for the trait Testosterone

| | | | | | | | | | RULE | |
|---|---|---|---|---|---|---|---|---|---|---|
| | | | | | | | | | VALUE | |
| | GENDER | | | RESULT | Logic 2 | | | | ADD/ | |
| | MALE/ | | | HIGH | AND/NOT/ | DATA | RESULT | RESULT | SUSBTRACT/ | |
| | FEMALE/ | DATA | RESULT | (IF APPLIC- | ANDNORT/OR/ | ELEMENT | LOW | HIGH | DIVIDE/ | RULE |
| RULE | BOTH | ELEMENT | LOW | ABLE) | ORNOT | 2 | 2 | 2 | MULTIPY | ACTION |
| 1 | FEMALE | SEX | F | F | | | | | | EXIT |
| 2 | MALE | [symbol] | [symbol] | [symbol] | | | | | [symbol] | EXIT |
| 3 | MALE | [symbol] | [symbol] | [symbol] | | | | | [symbol] | EXIT |
| 4 | MALE | [symbol] | [symbol] | [symbol] | | | | | [symbol] | EXIT |
| 5 | MALE | [symbol] | [symbol] | [symbol] | | | | | [symbol] | EXIT |
| 6 | MALE | [symbol] | [symbol] | [symbol] | | | | | [symbol] | EXIT |
| 7 | MALE | [symbol] | [symbol] | [symbol] | | | | | [symbol] | CONTINUE |
| 8 | MALE | [symbol] | [symbol] | [symbol] | | | | | [symbol] | CONTINUE |
| 9 | MALE | [symbol] | [symbol] | [symbol] | | | | | [symbol] | CONTINUE |
| 10 | MALE | [symbol] | [symbol] | [symbol] | | | | | [symbol] | CONTINUE |
| 11 | MALE | [symbol] | [symbol] | [symbol] | | | | | [symbol] | CONTINUE |
| 12 | MALE | [symbol] | [symbol] | [symbol] | | | | | [symbol] | CONTINUE |
| 13 | MALE | [symbol] | [symbol] | [symbol] | | | | | [symbol] | CONTINUE |
| 14 | MALE | [symbol] | [symbol] | [symbol] | | | | | [symbol] | CONTINUE |
| 15 | MALE | [symbol] | [symbol] | [symbol] | | | | | [symbol] | CONTINUE |
| 16 | MALE | [symbol] | [symbol] | [symbol] | | | | | [symbol] | CONTINUE |
| 17 | MALE | [symbol] | [symbol] | [symbol] | | | | | [symbol] | CONTINUE |
| 18 | MALE | [symbol] | [symbol] | [symbol] | | | | | [symbol] | CONTINUE |
| 19 | MALE | [symbol] | [symbol] | [symbol] | [symbol] | [symbol] | [symbol] | [symbol] | [symbol] | CONTINUE |
| 21 | MALE | [symbol] | [symbol] | [symbol] | [symbol] | [symbol] | [symbol] | [symbol] | [symbol] | CONTINUE |
| 24 | MALE | [symbol] | [symbol] | [symbol] | [symbol] | [symbol] | [symbol] | [symbol] | [symbol] | CONTINUE |
| 25 | MALE | [symbol] | [symbol] | [symbol] | [symbol] | [symbol] | [symbol] | [symbol] | [symbol] | CONTINUE |
| 27 | MALE | [symbol] | [symbol] | [symbol] | [symbol] | [symbol] | [symbol] | [symbol] | [symbol] | CONTINUE |
| 28 | MALE | [symbol] | [symbol] | [symbol] | | | | | [symbol] | CONTINUE |
| 29 | MALE | [symbol] | [symbol] | [symbol] | | | | | [symbol] | CONTINUE |
| 30 | MALE | [symbol] | [symbol] | [symbol] | | | | | [symbol] | CONTINUE |
| 31 | MALE | [symbol] | [symbol] | [symbol] | | | | | [symbol] | CONTINUE |
| 32 | MALE | [symbol] | [symbol] | [symbol] | | | | | [symbol] | CONTINUE |
| 33 | MALE | [symbol] | [symbol] | [symbol] | | | | | [symbol] | CONTINUE |
| 34 | MALE | [symbol] | [symbol] | [symbol] | | | | | [symbol] | CONTINUE |
| 35 | MALE | [symbol] | [symbol] | [symbol] | | | | | [symbol] | CONTINUE |
| 36 | MALE | [symbol] | [symbol] | [symbol] | | | | | [symbol] | CONTINUE |
| 37 | MALE | [symbol] | [symbol] | [symbol] | [symbol] | [symbol] | [symbol] | [symbol] | [symbol] | CONTINUE |
| 38 | MALE | [symbol] | [symbol] | [symbol] | [symbol] | [symbol] | [symbol] | [symbol] | [symbol] | CONTINUE |
| 39 | MALE | [symbol] | [symbol] | [symbol] | [symbol] | [symbol] | [symbol] | [symbol] | [symbol] | CONTINUE |
| 40 | MALE | [symbol] | [symbol] | [symbol] | [symbol] | [symbol] | [symbol] | [symbol] | [symbol] | CONTINUE |
| 41 | MALE | [symbol] | [symbol] | [symbol] | | | | | [symbol] | CONTINUE |
| 42 | MALE | [symbol] | [symbol] | [symbol] | | | | | [symbol] | CONTINUE |
| 43 | MALE | [symbol] | [symbol] | [symbol] | | | | | [symbol] | CONTINUE |
| 44 | MALE | [symbol] | [symbol] | [symbol] | | | | | [symbol] | CONTINUE |
| 45 | MALE | [symbol] | [symbol] | [symbol] | | | | | [symbol] | CONTINUE |
| 46 | MALE | [symbol] | [symbol] | [symbol] | | | | | [symbol] | CONTINUE |

[symbol]

indicates data missing or illegible when filed

Traits may not be exclusive, and may interact when being used to derive recommendations, or when modelling more complex biological functions or phenotypes.
Genetic data can be from more than one platform including 3rd party data sets.
Environmental data may encompass all other non-genetic data.

Once the trait scores for an individual have been calculated and utilised to understand the physiological makeup of the biological systems of the user, it may be possible to begin the recommendation process. Trait scores can be fixed at any given point in time, the fitness, diet plans given to the user are then based on the hierarchical compound weighting of traits associated with particular goal-linked questions and their effects on the biological systems to produce a readout figure. That is, the question (which defines a goal) has knowledge of which biological systems and traits are relevant to achieving the goal and the weighting and interactions between traits within the biological systems.

There may be various ways of defining the hierarchy of the traits and the interaction between them. Some of the methods for calculating the hierarchy include, but are not limited to, manually defining the hierarchy or computing said hierarchy by AI based upon collected data that has been analysed.

Different traits may affect different biological systems within the body and may affect more than one system at any one time. The system generally has knowledge about the compound effects and interactions any one or more trait can have on one or more body system for any one or more scenarios which may depend upon the goal of the user. The knowledge may be hardcoded, or may change and be refined over time, representing the current estimate based on the data currently analysed (include literature/AI based analysis). Further, the biological system can be characterised by a prediction calculation based on the traits of the user including the compounding effects of trait-trait interactions at the biological system level.

In such an embodiment, predefined and tailored recommendations and search methods based on what a user wants to achieve are used. Some trait scores and biological systems may not be relevant and thus may be excluded as the specific traits relevant may be based on the user's goals and thus, a selection within the system to pinpoint which traits and biological systems are relevant to the question may take place.

Accordingly, in this embodiment, which is complementary with earlier embodiments, the system or method can also determine one or more recommendations, said recommendations providing an actionable outcome for the user beyond the optimised workout plans, for example, diet or sleep recommendations. The recommendations can be determined based on one or more of: trait scores; and/or trait profiles; and/or data elements. Data elements, which will be described in greater detail below typically comprise one or more genetic factors, and/or one or more environmental factors and/or one or more goals.

Data elements are intended to provide extra resolution to the recommendations, but are not classed as traits in themselves but instead are context to a trait score. For example, based on a trait defining omega 6 intake (or omega 3 to omega 6 fat ratio) plus a data element describing diet the system or method can determine whether a recommendation is made for a user to alter their omega 6 intake. Due to the contribution of the data element ‘diet’, then the system can determine a sensible recommendation on how to do this based on their diet i.e. if they are vegetarian, a recommendation isn't made to eat more fish.

As an additional example, a recommendation may be provided to the user regarding the total amount of protein that should make up the users diet. This may be provided as a % of food intake, kcal intake, or a goal intake of protein in grams. The recommendation may be given to the user in bands, ranging from very low to very high. The recommendation level or band selected in this instance depends on the trait scores for the traits describing satiety, insulin sensitivity, obesity risk and lean body mass. Non-trait data elements that are relevant to determining the banding or level of recommendation for this example include age of the user, intermediate/active/ultimate goals of the user and goals of the selected fitness programme.

Although only two example recommendations have been described it can be appreciated that numerous recommendations could be generated.

In a further complementary embodiment, like with the trait scores and banding, the recommendations may be weighted depending upon the trait scores or trait bands or data elements which influence the recommendation. For example, a recommendation on whether to supplement the user's diet with vitamin B12 that is dependent on the trait describing folate metabolism and the environmental factor diet is likely to be heavily weighted to diet. It can be appreciated that other weightings may be appropriate for other recommendation. Bandings may also be used to highlight the relative strength or positively recommended effect of following the recommendations.

In another embodiment, when the overall system is queried as to what workout plan would meet a particular desired goal, an algorithm may be used to match workout plan with a diet plan based on the user's specifically defined biological system and trait profile, including the compounding effects of trait-trait interactions at the biological system level which will produce a trait profile in order to create recommendations for a user. To do this, questions are set up within the system 108. For each question, the system has the knowledge and the tools to identify the hierarchy between the traits and to determine which traits are relevant to the question.

This means the system may take the traits required for that specific question, and identify the interaction and hierarchy between them. For example, when recommending macronutrient splits, an individual's ‘resting blood triglycerides level’ and ‘resting blood glucose’ traits may impact on the recommendations for dietary fat percentage and carbohydrate percentage respectively. In addition, as these percentages of macronutrients contribute to an individual's overall calorie intake, these changes may impact on the relative amount of protein in the diet. Furthermore, another trait, ‘appetite suppression’, may also alter the protein percentage in the diet. This means that within the system, the interactions and hierarchies may be set out correctly for each question. This may then create a final set of scores from the traits (including the compounding effects of trait-trait interactions at the biological system level that will allow for the generation of the personalised recommendations).

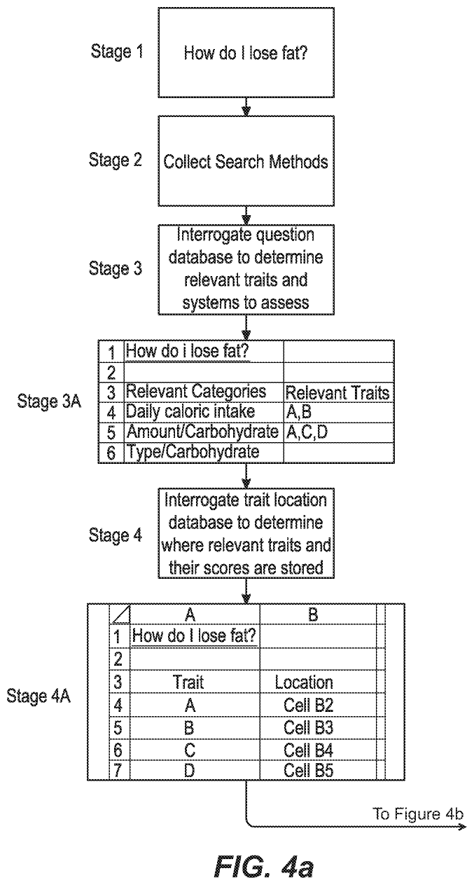

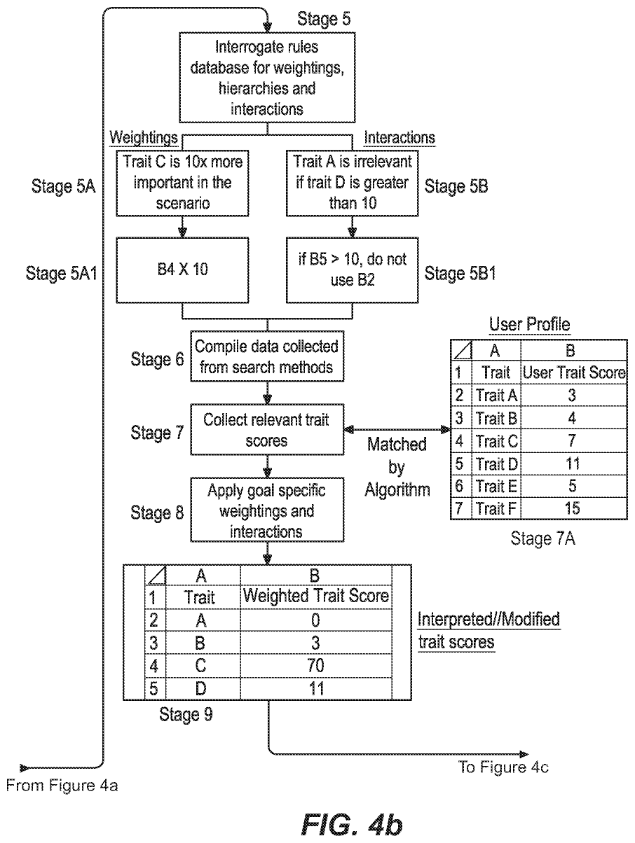

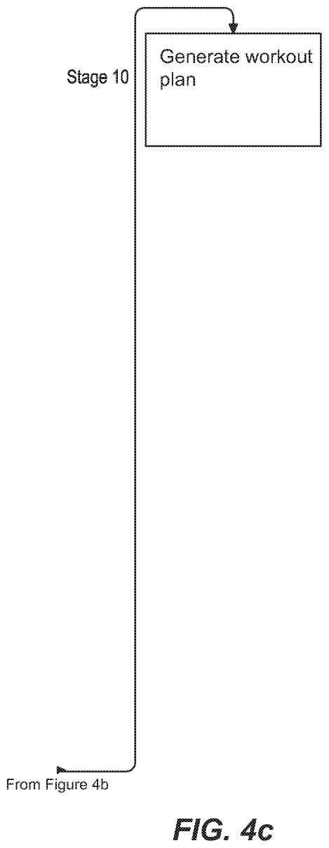

FIG. 4 depicts a specific example in which the system matches predefined goal-focussed questions with pre-defined health-related answers which have been hardcoded based on industry knowledge.

As shown in FIG. 4, the relevant stages for producing goal related fitness program for a specific user, based on their genetic makeup and environmental data may be detailed below.

In Stage 1, the user may decide upon the relevant goal they wish to pursue and select this fitness and diet related goal within the system. For example, a user may select “How do I lose fat?” For the purpose of this embodiment, we may define the following:
Search methods—data pertaining to:
   The relevant biological systems and traits to be assessed based on the goal
   The relevant weightings and interactions of those traits
   The location within the system of those user specific traits and thus, trait scores

Upon selecting that specific goal-related question, the system must be interrogated to extract the knowledge and search methods required to produce a diet and fitness recommendation for the goal-related question of the user.

Note: For the goal-related question in Stage 1, and other goal-related questions in other embodiments, the search methods may be predefined within the system prior to the question being asked. This means, the system may have predefined knowledge of all the search methods, for every possible question a user may be expected to ask of the system (or for questions provided to the user by the system) and thus, when a goal-related question is selected and the interrogation begins, the relevant database may output the specific search criteria. Tagging search methods to certain goal-related questions may be necessary as different goals may involve different traits, different interactions between the traits and be relevant to different biological systems of the user.

Each goal-related question is tagged with the necessary search information to find, order and prioritise relevant traits within the biological systems of the user. The system is instructed to begin gathering this information in Stage 2 of FIG. 4 where it starts collecting the goal-related, predefined search methods from their relevant databases. For the purpose of this embodiment, the search methods are located within the following databases and may be collected using a fetch algorithm:
Question database—containing relevant biological systems and traits
Trait location database—containing traits tagged with their specific locations within the user profile
Rules database—containing goal specific weighting and interactions to be applied

The question database, as shown in Stage 3A contains relevant recommendation categories that encompass the relevant biological systems and data stating which traits are relevant to each category for a specific goal-related question. For every goal-related question within the system, the question database within Stage 3A will contain the following:
List of relevant recommendation categories
List of traits that are relevant to that specific recommendation category

Note: The question database in Stage 3A may be produced based on industry knowledge, academia, 1st and 3rd party research and other sources. It provides a non-user-specific overview of which traits are relevant to which category for a specific goal-related question.

In this embodiment, the database has informed the system that the relevant categories that need to be assessed are “daily caloric intake”, and “carbohydrates” among others. For the purpose of this example, the categories to be assessed can be the “daily caloric intake” and “amount of carbohydrate” categories. Within the “daily caloric intake” category, the database has recommended that the traits that need to be assessed and are relevant to “losing fat” are traits A and B. Within the “amount of carbohydrate” category, the database has recommended that the traits that need to be assessed and are relevant to “amount of carbohydrate (to be consumed on daily basis in diet)” are traits A, C and D.

Note: the “daily caloric intake” and “amount of carbohydrate” categories and subsequent traits may not be the only relevant category to be interrogated for this specific goal, but for the purpose of this example embodiment, it is the only system computed and analysed here. A more thorough example of categories available is in Table 2 below.

In the example shown in Table 2, the category ‘daily calorie intake’ is associated with traits A and B. In this example, trait A corresponds to a user's resting metabolic rate, whilst trait B is the user's activity level(s). As noted above, the information for these traits may be obtained from external sources (such as a fitness/movement monitor for activity levels), external applications, questionnaires or the like.

For category 2: carbohydrates, there are a number of elements or variables on which recommendations can be made, such as the ‘amount’ or ‘type’ of carbohydrate. In this instance, with regard to the ‘amount’ of carbohydrate to be recommended, the traits ‘insulin sensitivity’ (C), ‘resting metabolic rate’ (A) and ‘blood triglycerides levels’ (D) are considered relevant. The traits relating to all relevant elements for each category make up the ‘trait profile’ for that category.

Once the system has knowledge of which categories and traits are relevant, it must then interrogate a trait location database—Stage 4—in order to obtain the necessary location information for a user's traits and in turn, trait scores. Location information, in this case, may be described as a specific point within the user profile that contains the relevant trait scores for the goal-related question.

The system needs to know where to fetch the trait scores from; this knowledge is passed to the system when it interrogates a trait location database in Stage 4A, again using a fetch algorithm. The system knows from Stage 3A which traits it must select, thus, when it has located the relevant

TABLE 2
Categories related to a goal/question of fat loss

| RECOMMENDATION | FAT LOSS | RELEVANT |
|---|---|---|
| CATEGORIES | RECOMMEND ON: | TRAITS |
| Daily Calorie Intake | Total calories | A, B, |
| Carbohydrates | Ratio and amounts, types, | C, A, D |
| | periodisation | |
| Fibre | Sources and importance for | Etc. |
| | weight loss | |
| Sat Fats | Ratio and amounts | |
| Mono Fats | Ratio and amounts | |
| Poly Fats | Ratio and amounts | |
| Total Fat | Ratio | |
| Protein | Ratio and amounts, timing of | |
| | intake | |
| Micronutrients | RDAs, food sources, vitamin | |
| | D/lactose/antioxidants | |
| Meal Frequency | Yes | |
| Caffeine | Impact on fat burning? | |
| Lactose | Advice on intolerance | |
| Hydration | Importance for fat loss and | |
| | recovery | |
| Sodium/Salt | | |
| Magnesium | | |
| EXERCISE | | |
| Type | Resistance and cardiovascular | |
| | (HIIT, steady state . . .) home | |
| | based | |
| Frequency | Days per week of resistance and | |
| | cardio, body splits | |
| Intensity | % VO2 max/HRmax | |
| Sets/Reps | Sets and reps for both HIIT, | |
| | resistance training | |
| Rest | between sets and workouts, | |
| | recovery strategies | |
| Duration | Specified cardio duration in | |
| | mins/interval durations, total | |
| | weeks | |
| Timing | Weekly and time of day | |
| SLEEP | Recommended amounts, | |
| | importance for fat loss, | |
| | strategies to improve sleep | |
| STRESS | | |
| SUPPLEMENTS | Caffeine, Green tea catechins | |

traits it collects and stores the traits (which are tagged with their locations in the user profile) to be used in the subsequent stages.

For the purpose of this embodiment, the trait location database shown in Stage 4A indicates that trait scores for traits A, B, C and D, the ones relevant to “losing fat”, are shown to be stored within the following locations within the user profile (shown in stage 7A):
Trait A—Cell B2
Trait B—Cell B3
Trait C—Cell B4
Trait D—Cell B5

Note: The system has detailed, quantified physiological knowledge and understanding of the user in the form of trait scores. These scores have been calculated based on the environmental and genetic data collected and are stored in a known location within the user profile. The trait location database in Stage 4A stores this location information for a user's trait scores.

Next, within Stage 5, the system must interrogate a rules database to obtain knowledge of the relevant goal-related trait weightings and interactions on the biological system level.

Note: That means, the specific weightings and interactions that need to be applied to Traits A, B, C or D for the “daily caloric intake” and “amount of carbohydrate” categories based on the question of “losing fat”.

In this embodiment, these weightings and interactions may be predefined.

For example, For daily caloric intake: Each trait has a score e.g. resting metabolic rate (G×E)=n1=high; activity levels (E×E)=n2=high and a set of logic/rules/algorithms exist that determine how to interpret each combination of scores together for each category on a biological systems level e.g. daily calorie intake=2500. This gives an output that is assigned to a particular outcome ‘band’ with associated recommendation e.g. eat 2500 calories a day.

Similarly, for amount of carbohydrate (as above): Trait C=(n3)=poor, Trait A=(n1)=high, Trait D=(n4)=high, e.g. the recommendation may be to reduce amount of carbohydrate in the user's diet relative to an average level by xx grams, where xx is the recommendation determined for the user.

Categories cannot be considered in isolation however, as there may be contradictions or contraindications and thus they must be considered in combination—the multi-level biological systems approach, exemplified in Stage 8.

For example, how does daily calorie intake impact on: carbohydrate vs protein vs fats, and how does carbohydrate amount impact on: daily caloric intake vs protein vs fats, etc.

For example, if calories are >2500, which may reflect an increased endurance activity level, the system can determine and advise the user to hold protein at a maximum level in terms of grams, and to increase carbohydrate ratio respectively to reflect the increases in calories required to support the increased activity.

In another example, if the output of ‘amount of carbohydrate’ in diet is ‘reduce’ and the output of ‘amount of fat’ in diet is ‘reduce’ the consequence on total macronutrient ratio is a significantly increased ‘amount of protein’ in diet. However this may not be an optimum health recommendation, as high protein can be linked to certain health issues. As a result, categories need to be prioritised appropriately. In this instance, the trait ‘insulin sensitivity’ may be considered to be more important than other relevant traits and as a result, the output for ‘amount of carbohydrate’ is prioritised above ‘amount of fat’.

For a trait relevant to a certain goal related question, a weighting may be applied to the trait score of a user. For example, there may be scenarios where, due to the goal, a specific trait score is required to be weighted more heavily within one or more categories. In this embodiment, as seen in Stage 5A, Trait C is seen to be 10× more important in the context of the “amount of carbohydrate” category for this goal-related question. Once the system has obtained that Trait C must be magnified by a factor of 10, it must also obtain the relevant code and/or equation for applying this weighting. This code may also be stored within the rules database and collected by the system in Stage 5A1.

For traits relevant to a certain goal related question, an interaction value may be applied to the trait scores of a user due to hierarchies within traits. For example, there may be scenarios where, due to the goal selected, a specific trait score becomes irrelevant due to a score of a different, or “overpowering” trait that takes priority. In this embodiment, as seen in Stage 5B, Trait A is seen to be irrelevant if Trait D has a larger value than 10 for this goal-related question. Once the system has obtained that Trait D must be ignored if Trait C is greater than 10, it must also obtain the relevant code and/or equation for applying this interaction. This code may also be stored within the rules database and collected by the system in Stage 5B1.

Upon completion of Stage 5, the system may compile all the search method criteria which can be used to model the relevant data based on the goal-related question, this is performed in Stage 6.

Once this has been compiled and computed, it may have the following knowledge and be able to progress to the modelling and recommendation stage:
Knowledge of which goal-related question the user has chosen—Stage 1
Knowledge of which recommendation categories and subsequent traits of the user are relevant to that goal-related question—Stage 3A—which it has gained from interrogating the question database in Stage 3.
Knowledge of where to obtain the relevant trait scores for the user—Stage 4 and 4A
Knowledge of the relevant goal-related trait weightings and interactions on the biological system level to be applied to the user's trait scores—Stage 5

Note: The user profile contains the trait scores along with a detailed physiological understanding of the user and the system now has a complete search methodology for the goal related question of “losing fat” as defined in the stages above.

In Stage 7, the system uses the search methods compiled in Stage 6 and fetches the relevant trait scores using a predefined algorithm from within cells B2, B3, B4 & B5 in the user profile—Stage 7A.

For this embodiment, these scores are:
Trait A=3
Trait B=4
Trait C=7
Trait D=11

The system stores these scores to be used in Stage 8 where the relevant weightings and interactions are to be applied.

Note: The trait scores detailed above for this user may be fixed at this given point in time as they are based on the environmental and genetic data collected, analysed and computed to produce the user profile. These trait scores will be used to apply the relevant goal-related weightings and interactions computed in Stage 6.

Note: The overall output may be based on the weightings of these trait scores along with the trait-trait interactions on a biological system level. These outputs will change relevant to the question and relevant to the initial trait scores of the user.

In this embodiment, using the user trait scores collected in Stages 7 and 7A, the specific weightings and interactions to trait scores A, B, C and D may be applied using an algorithm. This process occurs in Stage 8 of FIG. 9. These weightings may be dynamic and change dramatically depending on the goal of the user but for this case, the weighting and interactions for “losing fat” have been computed within Stage 5 of the system and the calculations are as follows:

Weighting:
Trait C×10=7×10=70

Interactions:
If Cell B5>10, Cell B2=0
Trait D in Cell B5=11,

Therefore: Trait A in Cell B2=0

Applying these weighting and interactions to the user's initial trait scores changes the value of the scores relevant to the search methodology computed for goal-related question of the user in Stages 3, 4 and 5. An algorithm may be used to apply the trait weightings and the trait-trait interactions as described above and then outputs a data set of interpreted/modified trait scores which the system can use to begin the recommendation process for diet and fitness plans. This output may be considered to be a trait profile that determines or defines the biological system level for that user. Such profile or biological system level is therefore a representation of the interaction between traits within categories e.g. the amount of carbohydrates recommendation linked to the metabolome and endocrine systems represented by the traits listed.

The interpreted and modified trait scores are stored within a database and this can be seen at Stage 9 of FIG. 4.

Note: In this embodiment, only interactions and weightings relevant to the goal-related question may be modelled. It may be noted that, multiple weightings and interactions may need to be applied for a specific goal-related question but for the purpose of this embodiment, a single weighting and a single interaction has been used. The algorithm may also search in between systems and analyse at the effects that systems have on each other at both system and trait level and these effects may further be used to modify a user's trait scores.

The system will now have user specific, goal-related interpreted/modified trait scores that encompass the relevant trait weightings and trait-trait interactions computed by the algorithm.

These scores are used to generate the optimised workout plan/fitness programme for the user at Stage 10, and may also be used to generate a diet plan to complement the fitness programme.

The progress of the user can be inputted back into the environmental data to allow the system to update the tailored recommendations. The diet and fitness recommendations may change any time a user updates or provides feedback to the system.

In addition to customer feedback, new genetic associations and research paradigms can be incorporated to constantly update the system. This may be done using bioinformatic analysis or artificial intelligence.

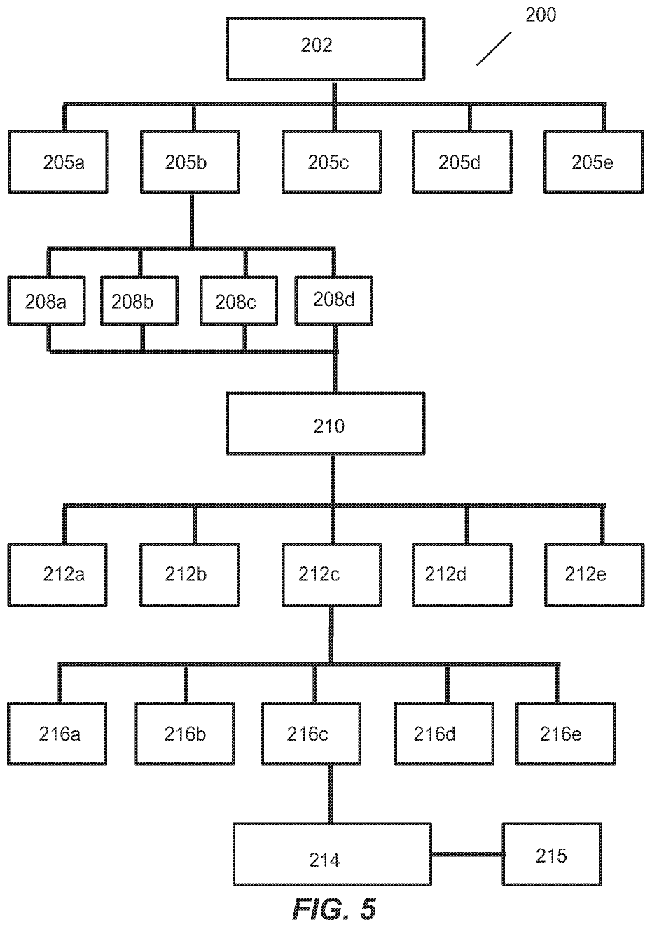

An additional complementary or alternative way of considering the system or method is described in relation to FIG. 5. Here, a method 200 of determining a fitness programme for a user is described. The method comprises the steps of determining goals 202 or additional information of the user. The goals or information can include questionnaires, genetic data, physiological data and environmental data of the type described in detail above in relation to the other figures. It can be appreciated that the system may obtain only a partial set of data at point 202 and may query and utilize goals or additional data at later points of the method, such as once the trait profile of a user is known.

Once the data or goals 202 are received or obtained, the method or system identifies a number of traits 205a-e, each trait being physiological, behavioural or biological states of the user that are potentially relevant to the goals 202. Again, examples of traits are described above. Traits may be identified based on the goals or data 202, or it can be appreciated that all or a selected ensemble of traits may be used.

Taking trait 205b as an example, the trait 205b has a number of genetic and/or environmental factors 208a-d that are relevant to the trait 205b. Some traits may have only genetic factors, some traits may have only environmental factors, although the majority of traits have a combination of genetic and environmental factors.

It can also be appreciated, that although shown as having four factors 208a-d, a trait may have more of less factors. Each factor typically provides a score, value or range that is indicative of the effect that the factor has on the trait.

As a specific example, for testosterone, genetic variations associated with the genes ESR1, ACTN3, SHBG1 and SHBG2 are considered relevant genetic factors, whilst body type, body fat %, age, activity levels, weekly active hours and sex are relevant environmental factors. The factors are obtained from the goals/data 202 obtained from the user.

Once the relevant traits 205 and their associated factors 208 are known a trait score is calculated 210. The trait score is an indication of the degree of expression of the trait for the user. Trait scores can be values or can be a series of bandings 212a-e depending upon the trait and/or the detail or complexity of the trait analysis required.

The trait scores are determined by weighting the genetic and/or environmental factors, where the weighting defines the likely effect that the genetic and/or environmental factors have on the trait. The weighting is determined by the system and it may be appreciated that different factors may have differing weightings depending upon the relative values of the other factors. For example, if a user's bodyfat is above a certain threshold value, then all genetic factors may be weighted sufficiently lower such that bodyfat is the key determinant for the trait score. Alternatively, if the user's bodyfat is below a threshold value, or within a bounded series of values then genetic factors may be a key determining factor. The nature of the weighting may be linear, but is often parabolic.

Unknown factors may be approximated based on the other factors, or may be assigned an average value based on other user's data. Similarly, in scenarios where specific values of traits are known, such as the result from a blood test for testosterone levels, then the factors can be disregarded and the actual result or reading used.

The trait score is indicative of a particular trait outcome. This is particularly easy to appreciate where bands are used. For example, a user's testosterone trait may be deemed very high, high, moderate, low or very low depending upon the calculated trait score.

A trait score 210 is calculated for each trait 205 that is under consideration by the system 200. Traits that do not contribute to the goals 202 of the user may be given a null or zero score. This is particularly the case for traits that are binary—the trait is either relevant or not. Once the trait scores for the traits 205 are known, a trait profile can be determined. The trait profile can be considered to be the user's health status or ‘biological avatar’ and provides an indication of the user's likely biological, physiological and/or behavioural response to a fitness programme.

Based on the trait profile, the system can then determine a fitness programme 214 that is tailored to achieving the goals of the user. The system may determine that the ultimate goals of the user require the user to first reach an alternative milestone or minimum level. For example, a minimum fitness level may be required to achieve some goals. Alternatively, the system may determine that a user's health or fitness would be better served by improving the user's cardiovascular health rather than attempting to build muscle (as an example).

The fitness programme 214 may, optionally but shown in this embodiment, be comprised of a number of recommendations 216a-216e as described earlier. The recommendations 216 are generally related to exercise (exercise type, frequency, etc.), but may also include nutritional (for example, daily calorie intake, protein ratio etc.), supplemental (Omega 3/6 supplementation, creatine supplementation etc.), wellness (# of hours sleep, etc.), or other categories of recommendations. Each recommendation 216 is based on a predetermined trait or combinations of traits, optionally with data elements 215.

As with traits, the recommendations 216 may be banded, or may be values. As an example, a recommended daily calorie intake is generally a value or number, whilst workout frequency will be generally provided to the user as a banding from low, medium, high, etc. with each band representing an action for the user optimised towards meeting the goals 202.

As noted above, data elements 215 may be utilized to add additional context and information to the workout plan or recommendations. Data elements 215 are typically one or more genetic factors, and/or one or more environmental factors and/or one or more goals.

It can be appreciated that the system and method 200 described are capable of being dynamically updated in semi-real time such that feedback on the user's response to recommendations 216 and the user's compliance with these, which may be self-reported or may be incoming data from a device or test aids development of the fitness programme 214 and additionally the traits 205 if the incident data 202 or goals of the user change.

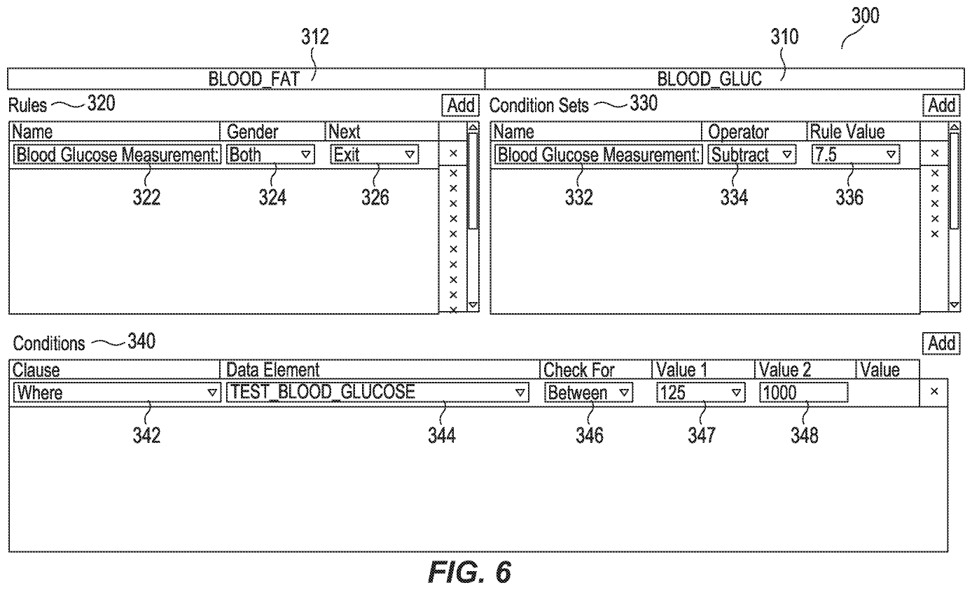

FIG. 6 shows an example of a user interface or user manual 300. The user interface 300 is intended to be a supplemental option to the embodiments described above. For example, the manual 300 may be implemented between boxes 107 and 108 in FIG. 1. The user interface provides an interface for 3^{rd} parties or power users to implement and create their own rules and conditions relating to traits and recommendations. This allows additional traits and recommendations to be determined, or existing traits modified accordingly. The user interface 300 has a browser based layout with tabs, buttons and drop down menus.

In the example shown in FIG. 6, a tab 310 is provided for the trait ‘BLOOD_GLUC’. This rule deals with measurements of resting blood glucose. A further tab 312 for the trait ‘BLOOD_FAT’ is currently unselected. Within the interface 300, a number of rules can be generated. New rules can be added by selecting an add button. In the present example, one rule 324 is shown. The rule relates to blood glucose measurement of the user. Rules may be selected from known rules, or may be created. The rule 324 is shown as applicable to both genders 326, with an additive being that the program is stopped from running if the rule conditions are met.

These rules 320 can be assigned gender, and be either additive (continue') or stop the program running (exit; this happens when a data element of a rule allows for full predictive capability at that point).

Condition sets 330 can also be defined for each rule 320. In this instance, the condition set is that if the rule 324, 332 is met, then a trait score is returned with an operator 334 of subtract (although addition, divide, multiply could apply) and a value 336 of 7.5. So the condition set has a value of −7.5 associated with it (subtract 7.5′). 1) The condition set allows for addition, subtraction multiplication or division between rule values.

Each condition set 330 can also have a number of conditions 340. In this instance, a logic operator or clause 342 informs the system that where the data element 344 (data element being data of the selected user obtained as noted above) has a value according to a check for (in this instance between) 346 two value bands 347, 348, then the condition assigns a value according to the condition set 330. So the condition will assign the value of −7.5 to a trait score when a user has a blood glucose test (Test _Blood_Glucose) between 125-1000. This is very high. The conditions can be qualitative information, or be values or ranges of values set that allow for addition, subtraction, multiplication or division between rule values

Values can also be assigned to genetic variants. For example, a value may be assigned if a user has a specific genotype. Traits can also have assigned bands that house ranges of trait scores. A trait band is a range of values that are assigned to one particular trait outcome—for example for ‘blood glucose’ ‘VERY_HIGH’. Values can also be used to assign more precision over time.

From this user interface 300, as user or customer data is fed into the system, the system will analyse each rule, with its condition sets and conditions, in turn. At the end a trait score will be calculated. Traits scores and where used trait bands can be used to build recommendations. Recommendations can also have assigned bands that will house ranges of recommendation scores. These can then be assigned to recommendations.

Traits and recommendations can be published to a ‘Vault’ (stored), to the ‘Live Server’ where they are used in customer products, to a ‘Test Server’ or sandbox where they can be tested for association, efficacy and consistency with existing or third party data sets, and to ‘Other Members’ of the system for analysis and expansion. The user interface 300 provides a powerful tool for analysing trait responses and aid understanding and compliance with workout plans/fitness programmes.

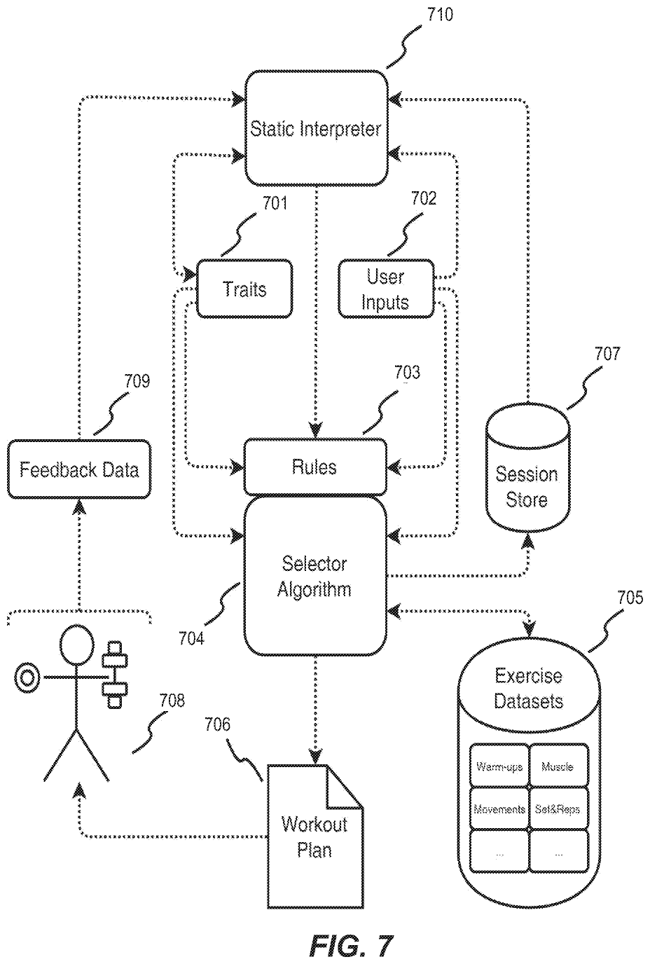

FIG. 7 depicts the system for generating an optimised workout plan in more detail. To initiate the generation of a personal work out plan, at least two sets of inputs must be provided to the system: a set of traits 701 associated with an individual, which are derived from the individual's genetic and environmental data as described above, and a set of user inputs 702 to direct construction of an appropriate workout plan.

As described above, traits 701 are biological descriptions of individuals that include both genetic and environmental factors. Traits define attributes of the user including those such as lactate clearance, endurance and power and each might be scored numerically or banded high/med/low for an individual. Traits help to describe the user's predicted response to certain exercises, and are used by the system to construct effective work out plans. The traits are organised into a trait profile of the individual, which provides an indication of the individual's physiological response to exercises of different types, durations, intensities etc.

User inputs 702 may include goals, for example fitness or weight loss goals, and other user work out condition. Goals include ‘Get Fit’ and ‘Build Muscle’. Further, conditions might include the location of training, equipment availability, type of cardio workout preference (e.g. running/cycling) and training day schedule. These user inputs help the system construct work out plans appropriate to the aspirations and preferences of the user. Further, it might be appreciated that any form of descriptive data associated with the user might be used to fuel the construction of personal workouts. This might include, but is not limited to, health records, social media profiles or bank details.

Traits and user input data are ingested by a rules base 703 which functions to guide the assembly of the complete workout routine of exercises. Using an iterative process, fuelled by the trait profile and user inputs, data associated with specific exercises is used to select appropriate and effective exercises and exercise programs from multiple exercise datasets 705.

This iterative selector-based fetch process is facilitated by a selector algorithm 704 with which the rules base is associated. The algorithm 704 coordinates the appropriate retrieval and transmission of data between the rules base 703 and exercise datasets a705 to fully populate personalised workout plans.

In the first step of this iterative process a high-level work out plan of specified length is constructed by the rules base 703 based purely on traits 701 including proficiency, power and endurance capability, frequency, recovery ability and lactate clearance and user input data 702, specifically a training schedule. The program does not contain specific exercises but defines work out days with categories of training to define day types from one or more of: strength development, strength stimulation, strength recovery, cardio stimulation, cardio development, and cardio recovery. Further, each day of the program is populated with appropriate warmup, workout and cooldown blocks. As such, a high-level workout routine is devised, already specific to the mosaic of traits and preferences for each user.

Using these data, in combination with trait and user inputs, the selector algorithm 704 then interrogates the tagged data held within banks of matrices in the exercise datasets 705. These datasets include descriptions of exercises (e.g. type of movement/resistance/equipment/muscle groups) as well as exercise parameters, e.g. sets and rep choices and warm up routines.

This fetch process is iterative, and retrieved exercise data is used in further interrogations of the exercise datasets 705. Each selection of a piece of data, a type of exercise, for example, has a compounding effect on the available choices for the following selections of data. This cycle continues until the entire work out plan is populated with appropriate sets of workouts which are appropriate and effective to the user's traits and inputs.

The completed optimised workout plan 706 is delivered to the user to direct their workout sessions 708 and a copy is provided in a separate database of workout sessions stores 707 associated with the user's identity for subsequent retrieval, if needed. The workout plan might be provided to the user in many different formats including, a mobile phone app, email, web browser, push notifications to a wearable device, or uploaded directly to exercise machines in gyms.

As the user progresses through their workout plan, intra-workout feedback may be provided in many ways, including telemetric through wearable devices, connect gym equipment, manual response to questionnaires or smart items, synced with the dynamic planner system 709.

In addition, the user may engage, or be prompted to engage with the dynamic planner pre- and post-work, providing additional feedback data that might be used to reconfigure their workout plan.

These data 709 are ingested to a separate set of rules held within a static interpreter 710. Using the context of traits 701, user inputs 702 and previous exercise history 707 the feedback data is interpreted to provide an extra set of inputs to the rules base 703 of the plan generator. The feedback data may also be used to update the trait profile of the individual based on empirical observations of the individual's response to exercise as measured by wearable fitness devices or other devices capable of generating data corresponding to the user's physiological condition.

Using the additional information provided by the static interpreter 710, the dynamic plan builder may iterate the process of plan building to provide the user with an up to date workout plan adjusted in line with the interpretation 710 of the feedback data 709.

In an alternative embodiment, the static interpreter 710 may be replaced by an AI interpreter module which can ingest feedback data 709 to interpret adjustments to the rules 703, selector algorithm 704, or the exercise datasets themselves 705 in the knowledge of traits 701, user inputs 702 and provided workout histories, held in the session store 707.

Moreover, the AI interpreter may ingest other personal data to guide plan adjustments. These data might include health records, historical wearable data, social media analysis, nutritional information and muscle biopsies.

Further, beyond the feedback data inputs 709 described in the previous embodiment, an AI-based adjustment may allow for a much greater spectrum of feedback data to be ingested. For example, forms of unstructured data, including sections of descriptive text generated from an AI workout chatbot, might be analysed in an AI-based system.

The AI interpreter 711 acts directly to adjust traits 701, plan construction rules 703, as well as the data held within the exercise dataset tables 705 themselves. In combination, feedback results in a fully adaptive plan builder, where both the algorithm that constructs workouts 703, the data that is fed into the system 701, 702 and tagged data that the system builds plans from 705 can be dynamically adjusted based on interpreted feedback.

Furthermore, it can be envisioned that personalising feedback may act both on individual plans and be harvested at a higher level to optimise parts of the dynamic plan builder system for all users.

In a specific embodiment of the system and method shown in FIG. 7, the user inputs 702 include:

goal=GETFIT
their goal is to get fit
(Options: get fit, lose weight, build muscle, get lean,)

location=GYM
they have access to gym equipment
(Options: gym, home)

cardio=RUN
they prefer to run over cycling
(Options: run, bike)

focus=NEUTRAL
Fortnight cycle, first week resistance training focussed, and second week cardio focussed. The other options are single week cycles.
(Options: resistance training, cardio, neutral)

selected training days per week=MONDAY, TUESDAY, WEDNESDAY, THURSDAY, FRIDAY AND SATURDAY
(Options: any sequence of 3 to 6 days per week)

optimal=FALSE
if true—the optimal weekly schedule for their plan and traits is selected for them and the selected training days per week is disabled

sex=MALE
(Options: male, female)

The trait profile 701 is derived in the manner described above, and includes at least the following physiological properties of the individual:

proficiency=ADVANCED
they would benefit from more intense training
(Options: GPP obese, obese, GPP beginner, beginner, intermediate, advanced)
proficiency is an output from the environmental data provided by the user, including activity levels, current frequency of participation and years of activity experience.

recovery=HIGH
their tendency is to recover quickly from exercise—between sets
(Options: very high, high, medium, low, very low)
Recovery is an output from: genetic factors: ACTN3, ACE and MCP, plus environmental data: body composition, age, sex, activity levels, current frequency of participation.

frequency=LOW
their tendency is to recover less quickly from exercise—between sessions.
In this scenario (LOW frequency), training fewer days per week will achieve better results than a week full of exercise with volume being equated.
(Options: high, medium, low)
Frequency is an output from: genetic factors: ACTN3, ACE, CKM, MCP, IL6 plus environmental data: body composition, age, sex, sleep quality, smoking, activity levels (in both endurance and resistance), current frequency of participation.

lactate clearance=MEDIUM
how quickly they remove lactate from the tissues after anaerobic exercise
(Options: high, medium, low)
Lactate clearance is an output from: genetic factors: MCP, plus environmental factors: activity levels (in both endurance and resistance), current frequency of participation

power=LOW
The propensity of one's bodies musculature to have a greater power capacity—including force output and fast twitch fibre recruitment.
(Options: high, medium, low)
Power is an output from: genetic factors: ACE, ACTN3, ADRB2, AGT, HIF1A, PPARA, PPARG, AMPD1, CKM, MTHFR, MTR, MTRR, MSTN, IGF1, NOS3, IL6, plus environmental factors: activity levels (in both endurance and resistance) and current frequency of participation.

endurance=HIGH
The propensity of one's bodies musculature to have a greater endurance capacity—including fatigue resistance and slow twitch fibre recruitment.
(Options: high, medium, low)
Endurance is an output from: genetic factors: ACE, ADBR2, BDKRB2, PPARA, VEGFA, UCP2, UCP3, AMPD1, CKM, PGC1A, HIF1A, plus environmental factors: activity levels (in both endurance and resistance)and current frequency of participation.

Once the genetic and environmental data have been received, along with the user inputs, and the trait profile has been generated, rules base 703 builds a schedule for a pre-determined length of time, e.g. 12 weeks, by matching a schedule to the selected training days per week and the user's traits, e.g. proficiency and frequency. The length of the schedule can be determined based on user inputs, alone, or may also take into account the user's trait profile and goals. Had optimal been set to true then it would have matched on the optimal training schedule for the traits proficiency and frequency.

Once the length of the plan has been determined, an empty plan is built for the length of time, e.g. 12 weeks, with, in this example, repeating fortnights as the user input focus=neutral. With other user inputs and other trait profiles, different repeating periods may be chosen. Each day within the repeating period is categorised according to a day type, which determines the type of exercises to be carried out on that day.

Below is the schedule repeating fortnight for the inputs:

Odd weeks:

Mon=StrengthStimulation
Focuses on strength speed and/or strength endurance, improving proprioception and movement proficiency

Tue=StrengthDevelopment
Focuses on improving submaximal strength levels and muscular hypertrophy development with resistance training protocols

Wed=CardioRegeneration
Recovery cardio protocols with exercises that have a low level of impact on the nervous system and soft tissue structures can be combined with additional core stability and postural exercise accessory movements

Thurs=StrengthStimulation
Focuses on strength speed and/or strength endurance, improving proprioception and movement proficiency

Fri=CardioStimulation
Cardio endurance maintenance to maintain levels of cardio fitness. Low level of impact on the nervous system or joints.

Sat=StrengthDevelopment
Focuses on improving the submaximal strength levels and muscle hypertrophy with resistance training protocols

Sun=Rest
Recovery

Even weeks:

Mon=StrengthDevelopment
Focuses on improving submaximal strength levels and muscular hypertrophy development with resistance training protocols

Tue=Cardio Stimulation
Cardio endurance maintenance to maintain levels of cardio fitness. Low level of impact on the nervous system or joints.

Wed=CardioDevelopment
To help develop the capacity to perform sustained cardio exercise at various levels of intensiveness. Looking to improve the maximal rate of oxygen consumption (V02 max)

Thurs=StrengthRegeneration
Recovery strength protocols using body weight and light free-weight exercises that have a low skill requirement in relation to the proficiency of the user.

Fri=CardioStimulation
Cardio maintenance to maintain levels of cardiorespiratory fitness with exercises that have a low level of impact on the nervous system and soft tissue structures

Sat=CardioDevelopment
To help develop the capacity to perform sustained cardio exercise at various levels of intensiveness. Looking to improve the maximal rate of oxygen consumption (V02 max)

Sun=Rest
Recovery day

Once each day in the whole schedule has been categorised into day types, the selector algorithm 704 identifies exercises and exercise parameters for each day according to the day type, trait profile 701 and user inputs 702. Each day can be considered to be a single session, and each session may include of a warmup, the actual workout and a cooldown, although warmups and cooldowns are not necessarily included.

Where warmups are included, the selector algorithm begins building the warmup by matching a set of warmup rules to the user's trait proficiency, the day type (e.g. StrengthStimulation etc.) and the week number. The rules may specify a number of rows that correspond to different exercises that are to be carried out.

These rules, along with the user's trait proficiency and location, are used to identify one or more exercises or movements from the exercise datasets 705. If more than one exercise/movement per warmup row is returned, one at random is chosen that does not match any movement in the main workout.

Once the exercise/movement have been identified for each row, the user's trait recommendations for recovery and lactate clearance are used to identify the exercise parameters for the identified exercises, i.e. the rest between exercises/sets, tempo to be used with the movement, and the effort level or rate of perceived exertion (RPE) required when performing the exercise or movement.

As the weeks increase the warmup exercises and sets and reps develop in difficulty and complexity.

The process operates in a similar manner for building the main exercises of the workout. The user's trait proficiency, the day's type (e.g. StrengthStimulation etc.) and whether the week is part of a specific training phase are used to query a group of workout blocks. A group may consist of 3 to 10 blocks which have a defined order, which defines a progression through the group as the user completes the exercises within the group, and may loop back to the beginning when completed.

A block comprises of a number of ordered rows ordered and grouped into sets. Each row defines a single exercise. Each set type modifies some of its exercise properties, for instance in a “straight” set the exercises are followed by their rests, in a “giant” set those rests are omitted.

Once the appropriate block has been selected, the process identifies one or more exercise parameters, e.g. sets and reps, for the block. There may be several different sets and reps tables for the workout, which have different filtering behaviour and looping over the plan's weeks. Which sets and reps table to use is controlled by the user's trait proficiency, and the day's type (e.g. StrengthStimulation etc.). Having selected the correct table, the rows are queried according to each of the exercises in the block and the user's traits for power and endurance. If any of the queries result in a single row of sets and reps then the row will be used to filter the movements. If not, a random movement is chosen.

A further rule, to fine tune random selection, is that an exercise cannot be repeated within in a session.

Using the user's traits recovery and lactate clearance and each movement's properties to filter for the rest and tempo to be used with that movement. If the sets and reps are not a single row for an exercise, then the associated movement's properties can be used to filter the sets and reps down to a single row.

Set and reps represent a progression of cycles anywhere from 5 cycles to 20. Properties on the row define the boundaries of the looping region. Longer plans may loop around these regions. The progression cycles change the exercise sets, reps, rest, RPE and tempo behaviour with week count, usually making the exercise harder. Rest and tempo usually modify the trait values slightly but they can also override them.

The building of cardio sessions follows a different pattern to that for strength sessions. For example, the warmup, workout and cooldown may all be defined together in the same row.

Cardio sessions may be divided into 2 types: main and lesser. The first cardio day in a week is the main, and any subsequent days are lesser. Main is the stressed session for the week for that day type and exhibits a different behaviour to lesser.

The user's trait proficiency, and the user inputs cardioOptions and focus and the day type are used to define a query to filter the cardio rules table to a single row, which defines the exercises that can be used and the order they are to run in over the plan's weeks, defining a progression. A cardio row also defines warmup exercise, its sets, reps, time and comments, the workout exercise group and the exercises, RPE and time for all the comprising exercises. Finally, it defines the cooldown exercise and the time.

For main sessions, the query is then updated by the cardio rule. If it is the first time the day type has been built, the query's progression index is set to 1. The query is then used to filter the cardio table down to a single row. Every time a day with the same type is built the progression index is incremented. When the value hits 3, the exercise is swapped for the next one in the cardio rule and the progression index set back to one. Once the end of the cardio rule has been reached it may loop back to the beginning with the progression index starting at 4 rather than 1, continuing randomly selecting from the cardio rule.

For lesser sessions, the exercise cannot be the same as the main in the same week. It uses the progression index from the beginning of the block i.e. 1 or 4 etc. It changes every week. If the day type is stimulation then the lesser exercise may always be light run for everyone. Other body types, such as GPP Obese, may use power walk instead.

Different types of cardio exercises may be used, for example running or cycling. For different cardio types, different rules may be followed to generate the sessions plan. For example, for cycling, a query may be built from the user's trait proficiency and user inputs cardioOptions and focus and the day type and used to filter of the cardio rows using the query, after which a single row from the remaining rows may be chosen randomly. After choosing a row, the difficulty of the exercise may be set according to the relevant progression index, which is increased every time an exercise is repeated.

After the optimised workout plans have been built and provided to users, feedback data 709 may be received from the users and used to modify each user's trait profile 701, the rules 703, selector algorithm 704, or the weightings used to determine users' trait profiles from their genetic data.

Feedback data 709 may be received by the system from a wearable device, such as a smart watch or fitness tracker in the case of direct physiological data, or also from a smartphone, tablet or computer where the feedback corresponds to GPS data or subjective assessments of the exercises that have been carried out.

The feedback data 709 itself may comprise data describing a physiological condition of the individual before, during, and/or after performing an exercise, for example a user's heart rate after an exercise has been completed.

The decision to modify the feedback data may be carried out in response to a comparison of the feedback data with a threshold. For example, where a user's trait profile indicates a specific heart rate recovery speed, the indicated heart rate recovery speed may be used to determine the threshold, and whether the heart rate recovery speed is above or below the threshold may be used to determine the response by the system.

Modification of the optimised workout plan in response to feedback data may be direct, e.g. by reducing the number of sets of a particular exercise where a user has failed to complete the prescribed number, or it may be indirect, e.g. by modifying a user's traits in response to a determination that their heart rate recovery speed is lower than their trait profile indicates it should be.

Taking the example of heart rate recovery speed, direct modification of the workout plan may include increasing the rest time between individual exercises or between sets. Indirect modification may include modification of the user's recovery trait, coupled with re-running the selector algorithm 704 to generate a new optimised workout plan based on the updated trait profile.

It will be appreciated that both direct and indirect feedback mechanisms may be employed by the system and in response to the same feedback data. The direct response, e.g. increasing rest time between sets, may be carried out immediately, during the current session, to adapt to the user's current condition, which may be due to environmental factors, e.g. illness, rather than the underlying genetic factors. The indirect response may be carried out over a longer term, for example if a user consistently shows poorer than expected heart rate recovery, indicating that its deviation from the expected value is unlikely to be the result of a transient environmental condition.

Several examples of feedback using feedback are discussed below, any of which may be employed in isolation or along with others as part of the present invention.

### Heart Rate Recovery

Faster heart rate recovery (HRR) after submaximal exercise can be a sign of functional overreaching so ensuring this only occurs for a short time period and is followed by a good recovery period can allow optimal use of it to induce ‘overcompensation’; considered a means to a successful performance increase.

Use Case 1): A user's heart rate, as measured by a wearable heartrate monitor, does not return to a value low enough after a set exercise. This happens for the same exercise for all sessions. The software responds by increasing the rest after this exercise is performed.

Use Case 2): A user's heart rate returns to a perceived low value faster than their traits indicated for all exercises. The software responds by reducing rests accordingly.

Use Case 3): A trait cohort (a population group who share the same ‘trait’) heart rate's return to normal quicker than expected. The information is fed back into the weighting system used to generate trait profiles, and new plans show an adjusted figure for that cohort.

### RPE—Rate of Perceived Exertion

Subjective user input data may be used to qualitatively assess the level of intensiveness being applied to an exercise by a user. For example, a target of 12-17 on a Borg RPE scale (0-20) during exercise or the simplified Borg CR10 (scale 0-10) target of 5-7 can imply the range at which users should be working out for resistance exercises. Above this level suggests working too hard which may lead to injury or overtraining. A drop below this range could indicate adaptation to load so a marginal increase in load could be prescribed for continuous improvements. This method of collecting data can work in conjunction with wearable devices to find out what metrics are associated with a user's performance levels. RPE feedback may also be used during a sessions to adjust the training load during the workout, and autoregulate the session volume.

Use Case: After a weight exercise, the user inputs the Kgs/lb's performed into a phone/wearable device application. They then indicate using the individual's subjective assessment (RPE) how they found the work. Algorithms determine from the heartbeat data during the exercise that they could do with a little persuasion in increasing the intensiveness at which they perform said exercise. In the subsequent week, the software increases or proposes to increase the weight marginally.

Emotional Data Input

Mood state questionnaires pre-workout session/post warm up preparation can be important indicators of burnout so when mood levels drop, training load should also be reduced for the daily session. This emotional data may be in response to prior training sessions and can be fed back into the system to adjust the weekly workouts structure or reduce the volume or intensity of the prior session to increase the total work performed over the course of the week.

### Blood Work

Blood work obtained from a user could identify deficiencies in key nutrients which could be impacting on training performance e.g. low levels of Vitamin B12 can lead to feeling lethargic so correspondingly they cannot reach the intensity desired during training or they miss sessions and reduce their total training volume.

### Medication

The ingestion of beta blockers reduces the exercise-induced heart rate levels, so this consideration allows us to alter a training program accordingly with this factored into the model. An increase in intensity/load when they are not ready for it can lead to negative repercussions. Users with ACE inhibitors should adhere to a regular cool down following exercise to prevent blood pressure dropping suddenly too low.

### GPS Monitoring & Weather Recognition

The environmental data gathered from a user's location could be used to adjust hydration and sodium level recommendations.

Use Case: A user indicates they dislike rain & cold weather when performing outdoor cardio activities. The software gets the weather information from their GPS location and provides an alert or adaptation to their training program to avoid prescribing the undesirable outdoor activity.

Conversely, if the weather is expected to be clement a bodyweight strength session could be performed outdoors.

### Menstrual Cycles

A female user's stage in the menstrual cycle can impact certain body compositional changes and hormonal factors that need to be considered. Excessive post exercise oxygen consumption can be experienced by females in the luteal phase of menstruation. Taking advantage of training methodologies such as High Intensity Interval Training sessions during this phase can increase energy expenditure of the user and aid body fat reduction. This phase of the menstrual cycle has also been associated with poor sleep quality; if this occurs the frequency of training and dietary recommendations can be adapted. This information paired with sleep data will provide a better scope of a user's sleep quality and recovery status.

### Behavioural Analysis

Some users (introverts) would prefer to follow fixed exercise selections for long periods of time because they like the ability to easily gauge progress, they enjoy the structure and predictability, and they like “finding their groove” with certain exercises. On the other hand, others (extroverted individuals) tend to “switch things up” more frequently because they feel stagnant if they use the same exercises on a regular basis for too long, and they like the soreness they get when they use an exercise they haven't performed in a while.

The use of personality typing/profiling to assess a user's neurodynamics and transmitter responses can be used to identify the optimal, most stimulating sequencing and programming of daily, weekly or monthly workout sessions.

Use Case 1): A user indicates they find the sessions boring with a series of negative emotional responses in questionnaires. The software updates the plan to increase variation, daily or weekly to increase adherence.

Use Case 2): A user indicates after a lapsed routine that they prefer a static routine. The plan is adjusted to remove variability of exercises and allow the user to feel more comfortable in something they can rely on to be the same week in week out.

### Exercise Selection

Allowing user exercise choice within the confines of variables that are unlikely to be detrimental to progress can help improve program adherence and progression. For example, you could have a fixed schedule for the priority exercises of the program, while allowing a user to choose which accessory exercises they perform on a week-to-week or session-to-session basis. This will increase adherence and the level of enjoyment experienced by the user in a gym setting. Also, if a user consistently ‘skips’ an exercise selection, the system can learn to avoid this selection in the future.

### Recovery & Fatigue

Allowing 24 to 72 hours' recovery after a specific muscle group or workout has been trained. This is dependent on the load level being used in the exercise, the traits associated with a user's recovery ability and external stress factors such as manual labour experienced by the user. A workout for highly trained advanced users with a high neural output would require at least 72 hours for recovery of certain muscle groups following a particularly heavy session (high RPE) or with an eccentric portion of exercise focus.

If a lower volume ‘rest’ day is required, we would reduce the total volume of a session by 40% compared to the heavier strength development days.

### Additional Training Modalities to a Program

If a user adds to their total workload of metabolic conditioning for the week, and their goal is to build lean muscle mass; for example, a very fast-paced cycle to work (high heart rate levels from wearables) then further conditioning work with a high neural output will need to be strategically manipulated to account for the added training volume of anaerobic work and to further manage accumulated fatigue levels

Exercise Progression and Regression

Exercise regressions for the same movement pattern can be optionally selected if the prescribed exercise in a workout places too much stress on a user in the gym setting (increasing cortisol levels, reducing adherence and positive performance/aesthetic changes). For example, if a front squat is prescribed for a user and is considered too difficult/too painful they would have access to similar regressed options for that movement such as a goblet squat or landmine squat to proceed to work on developing that movement pattern and have a balanced training routine.

### Test Markers

The use of ‘test’ sets can determine the progress of a user with a specific loading for an exercise. If they achieve repetition numbers higher than the recommended set repetitions, then we would advise an increase in the load selection for that exercise in the subsequent weeks. For example, if the user had a ‘test’ set and performed an excess of 2 reps above the rep target, then the system could advise increasing the weight by 5-10% the next time they come to perform that exercise. If they achieved the rep target by more than 3 reps, then the system would suggest for them to increase the weight by roughly 10-15% the following workout.

### Circadian Rhythms & Nutrition Timing

The understanding and reciprocal modelling of the interrelationships between health, lifestyle, sleep-wake cycles (and circadian rhythms), meal timings, and exercise may also be used to generate an optimal dynamic exercise plan. For example, the calculation of an individual's optimal sleep-wake cycle and meal timing structure will inform the best time to perform certain exercise types and intensities. This will allow for exercises to be performed at, for example, 1) the times that suit optimal performance for that individual, and 2) when an individual has had the optimal nutrition to support that performance; although other cases could be envisaged. It can also be conceived that individuals may deviate from their optimal courses of action, and so it is therefore required to measure these deviations in, for example, sleeping and eating, and alter future plans to generate a new optimal exercise plan. It is therefore possible to use wearable, or other data collection devices, to gather the health, lifestyle, sleep-wake cycle, meal timing data to both inform and support the daily makeup and structure of workout programs.

Use Case: Through monitoring heart rate and sleep-wake cycle harvested data, it seems that a trait cohort (population group that share the same ‘trait’) shows better than expected development when they exercise during the late morning. This information is then sent to relevant users (i.e. those that share the same trait) using their apps and emails.

Through monitoring heart rate, meal timing data and exercise timing data, it seems that a trait cohort show better than expected development when they eat immediately before exercise and 2 hours afterwards. Further examination indicates that the nutritional element and timing is very important for a user's development. This information is then sent to the relevant users using their apps and emails.

### Body Compositional Analysis

At different stages of the training program, the user may be prompted to input their current body stats, to determine whether they are making suitable and sustainable progress towards their goal. Every two to four weeks, a user would input their scale weight, their waist measurement with hip measurements being taken before they initiate the training plan. For example, if a reduction in body fat was the user's primary goal the target rate of loss would be 0.5-1% of bodyweight each week. If that goal wasn't accomplished the system would make alterations, for example, 1) the nutritional component makes a reduction in total caloric intake by 5-10% in the appropriate macronutrient's, 2) increase in the user's total energy expenditure with training plan adjustments. The system would similarly query the user to assess the causal issue to the stall in progress.

Use case: If the system indicates that the weight has plateaued on the scales and there hasn't been a progression towards the goal of the user, many measures are used to help find the appropriate response. The data gathered from a user's participation in exercise can determine if the training volume and intensiveness of the exercises were performed by the user and whether there has been a drop in performance measures.

The data gathered from their sleep cycle and heart rate will look at if they have had a negative sleep cycle, illness or stressful event which could impact their body compositional or dietary behaviours. Dietary factors will be then questioned to analyse if the user has ‘cheated’ on their diet or missed certain meals. Body stat analysis can be utilised to understand if their waist girths have decreased from the last check-in; it will similarly identify whether the stall in weight loss is due to an increase in lean body mass. The system will then prescribe, based on this data, the appropriate measures for the user to adopt including such actions as 1) increasing/reducing training volume or intensiveness, 2) increasing/reducing caloric intake 3) reassess in two weeks.

### Adherence Decline—Motivational Prompts

Users may experience a decline in adherence after a number of weeks actively involved on a certain regimen. One trait cohort of individuals has shown through collected data that the most prominent ‘drop-off’ point is after the fourth week of training compared to other cohorts. As such, a motivational notification will be sent to those users for encouragement purposes to increase adherence within the cohort. This notification may hold data showing their current improvement over the course of the regimen and display some information such as infographic data on their individual stats including improved resting heart rate, progression in body composition towards their goal and improved sleep quality.

Analysis of adherence between trait cohorts can help us identify the measures to use effectively to increase adherence. For example, one cohort of users will respond positively to in-app displays of improvement while the second group of users would respond more positively to emails. This information is then factored into the future interaction with users to improve successful goal achievement.

### Session Alterations

Use Case 1): Users may fail to complete a recommended session. This is then recognised by the system and a notification is displayed to the user to help them keep an account of the incomplete session. A few questions in an easy response format will be displayed to the user a few hours post session to identify the cause, and either adjust the plan, provide encouragement or advice for the next session.

Use Case 2): A user session has gone beyond the prescribed time by an extensive amount of time. A few questions in an easy response format will be displayed to the user a few hours post session to identify the cause, and either adjust the plan, provide encouragement or advice for the next session.

Use Case 3): A user goes on holiday for two weeks and requests the plan for those two weeks be recalculated for resistance training to display only bodyweight exercises and cardio to be swimming in replacement of running. The software will adjust their fortnightly program altering exercise volume prescription and type of exercises, for example, timed bodyweight circuits in replace of sets and reps for each exercise, and use the user's wearable data to measure the user's performance levels over the two weeks to feed back into the model to regulate for this time away from their original programming.

Use Case 4): A user is a student who goes home for the summer where they have limited access to a gym. They request the plan be adjusted for only one gym session per week and the rest to be cardio running and cycling. The software recalculates the plan for that period.

### Research Input

New research in genetics or corresponding fields can lead to more refined and adjusted trait recommendations amongst certain users or trait cohorts. The software will gradually field any alterations to a prescribed training plan gradually into the programming structure for the respective users.

Research gathered from our questionnaires across multiple formats could indicate that we propose an adjustment to a certain trait's weighting. The software will gradually field any alterations to a prescribed training plan gradually into the programming structure for the respective users.

Harvested data from a collection of users' data, or external/open source data, can be used to update the trait weightings that are used to generate trait profiles for all users. For example, from our analysis, if the feedback data shows that a build muscle plan produces marked improvements in cross-sectional muscle area muscle growth for a specific cohort or users who share a particular genetic variant when the frequency of program is increased in weeks ten to twelve, while the weekly training volume is still equated.

Feedback data 709 may also be received via a post-workout conversation with an AI system, for example via a person digital assistant or chatbot. The conversation is analysed by the AI system to determine:
The difficulty of the sessions perceived by the user (Rate of Perceived Exertion—RPE) eg. Volume, weight or exercises too difficult or too easy, for adjustments to subsequent sessions
The level of enjoyment after completion of this type of workout. If they enjoyed it keep this noted for the structure of the workout and the intensity level they enjoy, if they dislike then alter and see a future response.
The time it took to complete the session—are they under or overworking and is it within the timeframe we set. Collect data on the time they would like to complete a session and adapt future sessions to be more time efficient.
The physical characteristics displayed by the user i.e., whether they are out of breath. This can be used in conjunction with the session RPE to help get an accurate view of the sessions difficulty level.

Similarly, a pre-workout conversation would be used to determine:
How they feel prior to the session in terms of fatigue—additional daily stress factors (Rate of Perceived Recovery—RPR) e.g. If the user is tired, stressed, run down, or feeling exhausted from the previous session an adaptation can be made to the session to reduce overall training volume or intensiveness to accommodate for the fatigued state of the user.
Muscle Soreness e.g. If a muscle is outlined as being very sore from a previous session, the exercises could be altered to move away from placing too much stress in said area. This can also be a means of identifying any underlying movement issues, or patterning problems or potential injury occurrence if a muscle is continuously sore over a sustained period of the plan.
Available equipment if doing a home workout
the workout can be then adapted to fit the availability of the equipment.

An intra-workout conversation with an AI system could show:
The weight used for exercise—shows the level of intensity being experienced by the user. This would work in line with the RPR from the pre-workout chat.
Could they perform the prescribed exercise and its variables like sets, reps etc—if not, did they substitute, skip or adjust. This will help dictate exercise preferences, avoidances and the muscle groups which should get the most or least amount of stimulus during the program.
The information provided by the user could be used to help autoregulate the subsequent sessions, current session or adjust the training volume accordingly to help improve adherence, fatigue management and ensure a continued user progression.

From the feedback data 709 received from the user, the AI system is able to determine the workout plans that are working most effectively for certain population/groups e.g. obese, lean-male etc. This data can be used to profile the users into more granular groups which in turn would receive better results, as they will be matched to plans which were most successful with a group of people to which they align with.

The patterns of association from AI would help create groupings of users which have shown statistically that things are related. For example, HRV, sleep patterns and changes in physiological data based on changes in their habits. Collection of this information could help deduce the type of user who would benefit from this additional insight and refinement to their recommendations if they have shared traits, body statistics and self-selected data e.g. goal of building muscle.

Furthermore, the AI system could determine which aspects of workout plans brought about the most success for people within a particular grouping, and may also communicate with the users, for example, sending a “Keep it up” email if they are showing signs of ceasing to follow the workout plan.

In summary, the use of feedback data with a method for developing tailored workout plans based on an individual's genetic and physiological profile enables an optimised plan for the individual to be developed and refined. By comparison of an individual's feedback data with the feedback data of other individuals who share a genetic factor, improvements and insights can be obtained for new and existing individuals and can be used to generate further improved workout plans in future.

From reading the present disclosure, other variations and modifications will be apparent to the skilled person. Such variations and modifications may involve equivalent and other features which are already known in the art of health regime development, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom.

Throughout the description the terms “user” and “individual” have been used interchangeably to refer to the subject of the method. It will be appreciated that where the term “user” is used, this does not imply that the subject of the method for whom the optimised workout plan is generated is the person who necessarily interacts with any software of hardware operated as described herein. The direct user of the software or hardware may be a doctor or other healthcare practitioner, generating an optimised workout plan for a patient, who is the ultimate “user” as described herein.

The methods described above may be implemented as a computer program, which when run on a computer, causes the computer to configure any apparatus, including a circuit, controller, sensor, filter, or device disclosed herein or perform any method disclosed herein. The computer program may be a software implementation, and the computer may be considered as any appropriate hardware, including an analyser, a microprocessor, and an implementation in read only memory (ROM), erasable programmable read only memory (EPROM) or electronically erasable programmable read only memory (EEPROM), as non-limiting examples. The software implementation may be an assembly program. A user interface may be provided to allow for the inputting of information by the user—such interface may be a webpage, application or hardware based interface.

The computer program may be provided on a computer readable medium, which may be a physical computer readable medium, such as a disc or a memory device, or may be embodied as a transient signal. Such a transient signal may be a network download, including an internet download.

For the sake of completeness it is also stated that the term “comprising” does not exclude other elements or steps, the term “a” or “an” does not exclude a plurality, a single processor or other unit may fulfill the functions of several means recited in the claims and reference signs in the claims shall not be construed as limiting the scope of the claims.

1. A computer-implemented method of generating an optimised workout plan for an individual, the method comprising:
receiving genetic data, the genetic data describing a plurality of genetic factors of the individual;
receiving environmental data, the environmental data describing a plurality of environmental factors of the individual;
calculating a trait score for each of a plurality of traits based on the genetic data and environmental data;
defining a trait profile for the individual from the trait scores, said trait profile characterizing the user's physiological response to exercise;
generating an optimised workout plan for the individual by:
categorising days in a given time period into day types according to a training type and trait profile;
for each categorised day, identifying one or more exercises according to the day type and the trait profile;
for each identified exercise, determining one of more exercise parameters according to the individual's trait profile; and
compiling the categorised days, identified exercises and exercise parameters into an optimised workout plan covering the given time period; and
transmitting the optimised workout plan to the individual or a healthcare practitioner.

2. The computer-implemented method of claim 1, wherein the environmental data comprises a target number of days per week on which exercise takes place.

3. The computer-implemented method of claim 2, wherein categorising days in a given time period according to a training type comprises categorising each day according to the type and intensity of exercise to be carried out on the day.

4. The computer-implemented method of claim 1, wherein identifying one or more exercises comprises querying a database of exercises in which the exercises are categorised according to compatible traits and day types.

5. The computer-implemented method of claim 1, wherein, after the step of transmitting, the method further comprises:
receiving feedback data describing a physical condition of the individual;
modifying the optimised workout plan based on the feedback data; and
transmitting the modified optimised workout plan to the individual.

6. The computer-implemented method of claim 5, wherein modifying the optimised workout plan comprises modifying the exercise parameters.

7. The computer-implemented method of claim 5, wherein the feedback data corresponds to a physiological condition of the individual before, during, and/or after performing an exercise.

8. The computer-implemented method of claim 7, wherein modifying the optimised workout plan further comprises comparing the feedback data to a threshold and modifying the optimised workout plan based on whether the feedback data is above or below the threshold.

9. The computer-implemented method of claim 8, wherein modifying the optimised workout plan further comprises modifying the trait profile for the individual based on whether the feedback data is above or below the threshold.

10. The computer-implemented method of any one of claim 5, wherein the feedback data comprises heart rate data for the individual and exercise data such that the heart rate data describes the individual's heart rate before, during, and/or after the exercise described by the exercise data.

11. The computer-implemented method of claim 10, wherein the heart rate data indicates that the individual's heart rate decreases at a rate below a threshold after a set exercise.

12. The computer-implemented method of claim 11, wherein modifying the optimised workout plan comprises modifying the exercise parameters by increasing the rest time between exercises.

13. The computer-implemented method of any one of claim 10, wherein the heart rate data indicates that the individual's heart rate decreases at a rate above a threshold after a set exercise.

14. The computer-implemented method of claim 13, wherein modifying the optimised workout plan comprises modifying the exercise parameters by decreasing the rest time between exercises.

15. The computer-implemented method of any one of claim 11, wherein the threshold is set according to the trait profile.

16. The computer-implemented method of claim 15, wherein the threshold is set according to recovery and/or lactate clearance traits.

17. The computer-implemented method of any one of claim 11, further comprising updating the trait profile based on the received feedback data.

18. The computer-implemented method of any one of claim 5, wherein the feedback data is received from a wearable electronic device.

19. The computer-implemented method of claim 1, wherein the optimised workout plan is transmitted to one or more of: a mobile phone app, desktop app, tablet app, email address, web browser, wearable electronic device, and an exercise machine.

20. The computer-implemented method of claim 1, wherein the traits include—at least one item selected from the list consisting of: insulin sensitivity; obesity risk; gut microbiome profile; blood testosterone levels; dyslipidaemia, lactose intolerance, blood triglycerides level, blood glucose levels, oxidative muscle dominance, saturated fat level, satiety, folate metabolism, homocysteine levels, methionine levels, caffeine metabolism, hypertension levels, omega 6 intake or omega 3 to 6 ratio, circadian rhythm, sleep disturbance, trainable VO2 max, salt sensitivity, workout recovery between workout sessions, workout recovery during a workout session, lactate clearance levels, basal metabolism, lean body mass, endurance capability, power capability, conscious restraint, binge eating propensity, emotional eating propensity, eating behaviour and body fat.

21. The computer-implemented method of claim 1, wherein the genetic factors are genetic variants.

22. The computer-implemented method of claim 21, wherein the genetic variants include at least one item selected from the list consisting of: polymorphisms; insertions; deletions; gene copy number variants.

23. The computer-implemented method of claim 1, wherein the traits characterise biological systems of the user, said biological systems providing a representation of the user's physiological, behavioural and biological propensities and/or health status.

24. The computer-implemented method of claim 1, wherein calculating a trait score for each of the plurality of traits comprises:
identifying one or more of the genetic factors that are relevant to the trait;
identifying one or more of the environmental factors that are relevant to the trait;
assigning a weighting to each genetic and/or environmental factor, the weighting defining the effect that the genetic and environmental factors have on the trait; and
calculating a trait score for each trait based on the weightings and genetic and/or environmental factors.

25. The computer-implemented method of claim 24, further comprising:
receiving feedback data describing a physical condition of the individual;
comparing the feedback data of the individual with feedback data describing physical conditions of a group of individuals, wherein the group of individuals and the individual share a genetic factor;
updating the weighting assigned to the genetic factor based comparison; and
generating a new optimised workout plan for a new individual using the updated weighting.

26. The computer-implemented method of claim 1, wherein the exercise parameters include at least one item selected from the list consisting of: number of reps, number of sets, time between sets, rest time, weight to be lifted, distance to be run, distance to be cycled, speed to be run, speed to be cycled, time to run, time to cycle, interval length.

27. A data-processing system comprising:
means for receiving genetic data, the genetic data describing a plurality of genetic factors of the individual;
means for receiving environmental data, the environmental data describing a plurality of environmental factors of the individual;
means for calculating a trait score for each of a plurality of traits based on the genetic data and environmental data;
means for defining a trait profile for the individual from the trait scores, said trait profile characterizing the user's physiological response to exercise;
means for generating an optimised workout plan for the individual by:
categorising days in a given time period into day types according to a training type and trait profile;
for each categorised day, identifying one or more exercises according to the day type and the trait profile;
for each identified exercise, determining one of more exercise parameters according to the individual's trait profile;
compiling the categorised days, identified exercises and exercise parameters into an optimised workout plan covering the given time period; and
means for transmitting the optimised workout plan to the individual or a healthcare practitioner.

28. A non-transitory machine readable medium, having stored thereon instructions for performing a method of generating an optimised workout plan for an individual, comprising machine executable code which when executed by at least one machine, causes the machine to:
receive genetic data, the genetic data describing a plurality of genetic factors of the individual;
receive environmental data, the environmental data describing a plurality of environmental factors of the individual;
calculate a trait score for each of a plurality of traits based on the genetic data and environmental data;
define a trait profile for the individual from the trait scores, said trait profile characterizing the user's physiological response to exercise;
generate an optimised workout plan for the individual by:
categorising days in a given time period into day types according to a training type and trait profile;
for each categorised day, identifying one or more exercises according to the day type and the trait profile;
for each identified exercise, determining one of more exercise parameters according to the individual's trait profile; and
compiling the categorised days, identified exercises and exercise parameters into an optimised workout plan covering the given time period; and
transmit the optimised workout plan to the individual or a healthcare practitioner.
